SEQUENCE LISTING (6 sequences)

SEQ ID NO: 1 - protein, 405 aa, Homo sapiens
MPLLLYTCLLWLPTSGLWTVQAMDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDAELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 2 - protein, 383 aa, Homo sapiens
MDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDAELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 3 - DNA, 1510 bp, Homo sapiens
atccaccgcaggcttactgtacacatgctagggtccaggacagcaggaccaagccagcagaaacagcctgagcccaccgcagactggcctggctatactggacaatgccactcctcctgtacacctgtcttctctggctgcccaccagcggcctctggaccgtccaggccatggatcctaacgctgcttatgtgaacatgagtaaccatcaccggggcctggcttcagccaacgttgactttgccttcagcctgtataagcacctagtggccttgagtcccaaaaagaacattttcatctcccctgtgagcatctccatggccttagctatgctgtccctgggcacctgtggccacacacgggcccagcttctccagggcctgggtttcaacctcactgagaggtctgagactgagatccaccagggtttccagcacctgcaccaactctttgcaaagtcagacaccagcttagaaatgaccatgggcaatgccttgtttcttgatggcagcctggagttgctggagtcattctcagcagacatcaagcactactatgagtcagaggtcttggctatgaatttccaggactgggcaacagccagcagacagatcaacagctatgtcaagaataagacacaggggaaaattgtcgacttgttttcagggctggatagcccagccatcctcgtcctggtcaactatatcttcttcaaaggcacatggacacagccctttgacctggcaagcaccagggaggagaacttctatgtggacgagacaactgtggtgaaggtgcccatgatgttgcagtcgagcaccatcagttaccttcatgacgcggagctcccctgccagctggtgcagatgaactacgtgggcaatgggactgtcttcttcatccttccggacaaggggaagatgaacacagtcatcgctgcactgagccgggacacgattaacaggtggtccgcaggcctgaccagcagccaggtggacctgtacattccaaaggtcaccatctctggagtctatgacctcggagatgtgctggaggaaatgggcattgcagacttgttcaccaaccaggcaaatttctcacgcatcacccaggacgcccagctgaagtcatcaaaggtggtccataaagctgtgctgcaactcaatgaggagggtgtggacacagctggctccactggggtcaccctaaacctgacgtccaagcctatcatcttgcgtttcaaccagcccttcatcatcatgatcttcgaccacttcacctggagcagccttttcctggcgagggttatgaacccagtgtaagagaccacccacccagagcctcagcactgtctgactttgggaaccagggatcccacagaaatgttttggagagcgggaggtttcccccaatctcctccaagttcttctccctccaaccagagttgtgtctaactttaggcatcttttaataaatgtcattgcgactctgaaaaaaaaaaaaaaaaaaa

SEQ ID NO: 4 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 5 - protein, 238 aa, Homo sapiens
IVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 6 - DNA, 1020 bp, Homo sapiens
gggagaggaagtggagggcgctggccggccgtggggcaatgcaacggcctcccagcacagggctataagaggagccgggcgggcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaaaaaaaaaaaaaaaa

(19) United States
(12) Patent Application Publication
(10) Pub. No.: US 2018/0156823 A1
(43) Pub. Date: Jun. 7, 2018

(54) METHODS OF DETERMINING RISK OF CARDIAC ARRHYTHMIA

(71) Applicant: Pharmasan Labs, Inc., Osceola, WI (US)

(72) Inventors: Gustav F. Jirikowski, Osceola, WI (US); Gottfried H. Kellermann, Osceola, WI (US)

(21) Appl. No.: 15/830,886

(22) Filed: Dec. 4, 2017

Related U.S. Application Data
(60) Provisional application No. 62/429,449, filed on Dec. 2, 2016.

(51) Int. Cl. G01N 33/68; G01N 33/74; G01N 33/573; G16H 50/30

(52) U.S. Cl. CPC G01N 33/6893; G01N 33/743; G01N 33/573; C12Y 304/21037; G16H 50/30; G16H 10/60; G01N 2800/326; G01N 2333/8121; G01N 2333/96433; G01N 2800/52; G01N 2800/50

(57) ABSTRACT

Provided herein are methods of determining a subject's risk of developing a cardiac arrhythmia, methods of treating a subject having or at risk of developing a cardiac arrhythmia, methods of selecting a treatment for a subject having or at risk of developing a cardiac arrhythmia, methods of selecting a subject for administration of a treatment for reducing risk of developing a cardiac arrhythmia, methods of determining the efficacy of a treatment in a subject having or at risk of developing a cardiac arrhythmia, and methods of monitoring a subject having or at increased risk of developing a cardiac arrhythmia based on a level of one or more of glucocorticoid (GC), corticosteroid-binding globulin (CBG), and neutrophil elastase (NE). Also provided are kits that include two or more of an antibody that binds specifically to GC, an antibody that binds specifically to GBC, and an antibody that binds specifically to NE.

### CROSS-REFERENCE TO RELATED APPLICATION

This application claims priority to U.S. Provisional Patent Application Ser. No. 62/429,449, filed Dec. 2, 2016, the contents of which are incorporated herein by reference in their entirety.

### TECHNICAL FIELD

This invention relates to methods of molecular medicine and molecular biology.

### BACKGROUND

Glucocorticoids (GCs) are a class of corticosteroids that elevate serum glucose levels by stimulating gluconeogenesis in the liver, but they also increase glucose utilization in numerous other cells. GC levels are increased through activation of the hypothalamo-pituitary-adrenal axis upon stress response. GCs bind to the glucocorticoid receptor (GCR), and have a variety of effects on their target cells. Cytosolic GCR is dimerized upon binding GCs, and is translocated into the nucleus where GCR-GC acts as a transcription factor.

Corticosteroid-binding globulin (CBG) is a highly conserved 52 kDa glycoprotein with high affinity for adrenal steroid hormones. CBG is expressed in all mammalian species including humans and is structurally related to the family of serine protease inhibitors (SERPINs). The liver expresses the greatest levels of CBG under the transcriptional control of systemic estrogen and glucocorticoid levels. CBG is thought to play a significant role in systemic inflammation.

GCs block cytokine release from leukocytes via rapid membrane-mediated effects. Many leukocytes, including neutrophils, express a specific neutrophil elastase (NE) that lowers CBG affinity for GCs, thus liberating GCs from binding CBG at the cell membrane.

### SUMMARY

The present invention is based, at least in part, on the discovery that serum levels of CBG are decreased in subjects at risk of developing a cardiac arrhythmia. The present invention is also based, at least in part, on the discovery that serum levels of GC are elevated in subjects at risk of developing a cardiac arrhythmia. The present invention is also based, at least in part, on the discovery that serum levels of NE are elevated in subjects at risk of developing a cardiac arrhythmia. In view of this discovery, provided herein are methods of determining a subject's risk of developing a cardiac arrhythmia, methods of treating a subject having or at risk of developing a cardiac arrhythmia, methods of selecting a treatment for a subject having or at risk of developing a cardiac arrhythmia, methods of selecting a subject for administration of a treatment for reducing risk of developing a cardiac arrhythmia, methods of determining the efficacy of a treatment in a subject having or at risk of developing a cardiac arrhythmia, and methods of monitoring a subject having or at increased risk of developing a cardiac arrhythmia. In some embodiments, methods disclosed herein include performing an immunoassay to determine the levels of one or more of CBG CG and NE in a subject. In some embodiments, methods provided herein can be used to assess a subject's risk of developing a cardiac arrhythmia long before structural damage to myocardium occurs (e.g., before an increase of systemic troponin C levels), thus allowing for an earlier and more effective therapeutic intervention. Also provided herein are kits that include an antibody that binds specifically to CBG an antibody that binds specifically to CG and an antibody that binds specifically to NE.

Provided herein are methods of determining a subject's risk of developing a cardiac arrhythmia that include: (a) one or more of: performing an immunoassay to determine the level of CBG in a sample including blood, plasma, or serum obtained from a subject; performing an immunoassay to determine the level of GC in a sample including blood, plasma, or serum obtained from the subject; and performing an immunoassay to determine the level of NE in a sample including blood, plasma, or serum obtained from the subject; (b) comparing the determined level(s) of one or more of CBG GC, and NE to reference level(s) of CBG GC, and NE, respectively; and (c) identifying a subject having one or more of a determined level of CBG that is decreased as compared to a reference level of CBG a determined level of GC that is elevated as compared to a reference level of GC, and a determined level of NE that is elevated as compared to a reference level of NE as having an increased risk of developing a cardiac arrhythmia; or identifying a subject having one or more of a determined level of CBG that is about the same or elevated as compared to a reference level of CBG a determined level of GC that is about the same or decreased as compared to a reference level of GC, and a determined level of NE that is about the same or decreased as compared to a reference level of NE as having a decreased risk of developing a cardiac arrhythmia. In some embodiments, step (c) includes identifying a subject having one or more of a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia. In some embodiments, step (c) includes identifying a subject having two or three of a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia. In some embodiments, step (c) includes identifying a subject having a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia. In some embodiments, step (a) includes determining the level of one of CBG, GC, and NE in the sample. In some embodiments, step (a) includes determining the level of two of CBG, GC, and NE in the sample. In some embodiments, step (a) includes determining the level of CBG, GC, and NE in the sample. In some embodiments, step (c) includes identifying a subject having one or more of a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia. In some embodiments, step (c) includes identifying a subject having two or three of a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia. In some embodiments, step (c) includes identifying a subject having a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia.

Also provided herein are methods of determining a subject's risk of developing a cardiac arrhythmia that include identifying a subject determined to have one or more of a level of CBG that is decreased as compared to a reference level of CBG, a level of GC that is elevated as compared to a reference level of GC, and a level of NE that is elevated as compared to a reference level of NE as having an increased risk of developing a cardiac arrhythmia; or identifying a subject determined to have one or more of a level of CBG that is about the same or elevated as compared to a reference level of CBG, a level of GC that is about the same or decreased as compared to a reference level of GC, and a level of NE that is about the same or decreased as compared to a reference level of NE as having a decreased risk of developing a cardiac arrhythmia. In some embodiments, such methods include identifying a subject determined to have two or three of a level of CBG that is decreased as compared to a reference level of CBG, a level of GC that is elevated as compared to a reference level of GC, and a level of NE that is elevated as compared to a reference level of NE as having an increased risk of developing a cardiac arrhythmia. In some embodiments, such methods include identifying a subject determined to have one or more of a level of CBG that is decreased as compared to a reference level of CBG, a level of GC that is elevated as compared to a reference level of GC, and a level of NE that is elevated as compared to a reference level of NE as having an increased risk of developing a cardiac arrhythmia. In some embodiments, such methods include identifying a subject determined to have two or three of level of CBG that is about the same or elevated as compared to a reference level of CBG, a level of GC that is about the same or decreased as compared to a reference level of GC, and a level of NE that is about the same or decreased as compared to a reference level of NE as having a decreased risk of developing a cardiac arrhythmia. In some embodiments, such methods include identifying a subject determined to have two or three of level of CBG that is about the same or elevated as compared to a reference level of CBG, a level of GC that is about the same or decreased as compared to a reference level of GC, and a level of NE that is about the same or decreased as compared to a reference level of NE as having a decreased risk of developing a cardiac arrhythmia.

In some embodiments, methods of determining a subject's risk of developing a cardiac arrhythmia further include recording the subject's risk of developing a cardiac arrhythmia in the subject's clinical record. In some embodiments, the subject's clinical record is a computer readable medium.

Also provided herein are methods of treating a subject that include: (a) one or more of: performing an immunoassay to determine the level of CBG in a sample including blood, plasma, or serum obtained from a subject; performing an immunoassay to determine the level of GC in a sample including blood, plasma, or serum obtained from the subject; and performing an immunoassay to determine the level of NE in a sample including blood, plasma, or serum obtained from the subject; (b) comparing the determined level(s) of one or more of CBG, GC, and NE to reference level(s) of CBG, GC, and NE, respectively; (c) selecting a subject having one or more of a determined level of CBG that is decreased as compared to a reference level of CBG a determined level of GC that is elevated as compared to a reference level of GC, and a determined level of NE that is elevated as compared to a reference level of NE; and (d) administering a treatment for reducing risk of developing a cardiac arrhythmia to the selected subject. In some embodiments, step (a) includes determining the level of one of CBG, GC, and NE in the sample. In some embodiments, step (a) includes determining the level of two of CBG, GC, and NE in the sample. In some embodiments, step (a) includes determining the level of CBG, GC, and NE in the sample. In some embodiments, step (c) includes selecting a subject having two or three of a determined level of CBG that is decreased as compared to a reference level of CBG a determined level of GC that is elevated as compared to a reference level of GC, and a determined level of NE that is elevated as compared to a reference level of NE. In some embodiments, step (c) includes selecting a subject having a determined level of CBG that is decreased as compared to a reference level of CBG a determined level of GC that is elevated as compared to a reference level of GC, and a determined level of NE that is elevated as compared to a reference level of NE.

Also provided herein are methods of treating a subject that include selectively administering to a subject determined to have one or more of a level of CBG that is decreased as compared to a reference level of CBG a level of GC that is elevated as compared to a reference level of GC, and a level of NE that is elevated as compared to a reference level of NE, a therapeutically effective dose of a treatment for reducing the risk of a cardiac arrhythmia. In some embodiments, such methods include selectively administering to a subject determined to have two or three of a level of CBG that is decreased as compared to a reference level of CBG a level of GC that is elevated as compared to a reference level of GC, and a level of NE that is elevated as compared to a reference level of NE, a therapeutically effective dose of a treatment for reducing the risk of a cardiac arrhythmia. In some embodiments, such methods include selectively administering to a subject determined to have a level of CBG that is decreased as compared to a reference level of CBG a level of glucocorticoid GC that is elevated as compared to a reference level of GC, and a level of NE that is elevated as compared to a reference level of NE, a therapeutically effective dose of a treatment for reducing the risk of a cardiac arrhythmia.

In some embodiments of any of the methods described herein, a treatment for reducing risk of developing a cardiac arrhythmia is selected from the group of: a selective serotonin reuptake inhibitor (SSRI), a norephinephrine reuptake inhibitor (NRI), a dopamine reuptake inhibitor (DRI), a serotonin/norephinephrine reuptake inhibitor (SNRI), a norepinephrine/dopamine reuptake inhibitor (NDRI), a triple reuptake inhibitor (TRI), an anti-clotting medication, a heart rate management medication, a heart rhythm management medication, a blood pressure management medication, a neutrophil elastase inhibitor, a cholesterol management medication, a cortisol management medication, and a sympathetic activity management medication. In some embodiments of any of the methods described herein, a treatment for reducing risk of developing a cardiac arrhythmia can include performing increased monitoring of the subject or performing a surgical procedure on the subject.

Also provided herein are methods of selecting a treatment for reducing risk of developing a cardiac arrhythmia for a subject that include: (a) one or more of: performing an immunoassay to determine the level of CBG in a sample including blood, plasma, or serum obtained from a subject; performing an immunoassay to determine the level of GC in a sample including blood, plasma, or serum obtained from the subject; and performing an immunoassay to determine the level of NE in a sample including blood, plasma, or serum obtained from the subject; (b) comparing the determined level(s) of one or more of CBG, GC, and NE to reference level(s) of CBG, GC, and NE, respectively; and (c) selecting a treatment for reducing risk of developing a cardiac arrhythmia for a subject having one or more of a determined level of CBG that is decreased as compared to a reference level of CBG, a determined level of GC that is elevated as compared to a reference level of GC, and a determined level of NE that is elevated as compared to a reference level of NE. In some embodiments, step (a) includes determining the level of one of CBG, GC, and NE in the sample. In some embodiments, step (a) includes determining the level of two of CBG, GC, and NE in the sample. In some embodiments, step (a) includes determining the level of CBG, GC, and NE in the sample. In some embodiments, step (c) includes selecting a treatment for reducing risk of developing a cardiac arrhythmia for a subject having two or three of a determined level of CBG that is decreased as compared to a reference level of CBG, a determined level of GC that is elevated as compared to a reference level of GC, and a determined level of NE that is elevated as compared to a reference level of NE. In some embodiments, step (c) includes selecting a treatment for reducing risk of developing a cardiac arrhythmia for a subject having a determined level of CBG that is decreased as compared to a reference level of CBG a determined level of GC that is elevated as compared to a reference level of GC, and a determined level of NE that is elevated as compared to a reference level of NE. In some embodiments, the immunoassay used to determine the level of CBG is an enzyme-linked immunosorbent assay. In some embodiments, the immunoassay used to determine the level of GC is an enzyme-linked immunosorbent assay. In some embodiments, the immunoassay used to determine the level of NE is an enzyme-linked immunosorbent assay.

Also provided herein are methods of selecting a treatment for reducing risk of developing a cardiac arrhythmia for a subject that include selecting a treatment for reducing risk of developing a cardiac arrhythmia for a subject determined to have one or more of a level of CBG that is decreased as compared to a reference level of CBG a level of GC that is elevated as compared to a reference level of GC, and a level of NE that is elevated as compared to a reference level of NE. In some embodiments, the method includes selecting a treatment for reducing risk of developing a cardiac arrhythmia for a subject determined to have two or three of a level of CBG that is decreased as compared to a reference level of CBG, a level of GC that is elevated as compared to a reference level of GC, and a level of NE that is elevated as compared to a reference level of NE. In some embodiments, the methods include selecting a treatment for reducing risk of developing a cardiac arrhythmia for a subject determined to have a level of CBG that is decreased as compared to a reference level of CBG, a level of GC that is elevated as compared to a reference level of GC, and a level of NE that is elevated as compared to a reference level of NE.

Some embodiments of any of the methods described herein further include recording the selected treatment for the subject in the subject's clinical record. In some embodiments, the subject's clinical record is a computer readable medium.

In some embodiments of any of the methods described herein, the treatment for reducing risk of developing a cardiac arrhythmia is selected from the group consisting of: a SSRI, a NRI, a DRI, a SNRI, a NDRI, a TRI, an anti-clotting medication, a heart rate management medication, a heart rhythm management medication, a blood pressure management medication, a neutrophil elastase inhibitor, a cholesterol management medication, a cortisol management medication, and a sympathetic activity management medication. In some embodiments of any of the methods described herein, the treatment for reducing risk of developing a cardiac arrhythmia can include performing increased monitoring of the subject or performing a surgical procedure on the subject.

Also provided herein are methods of selecting a subject for administration of a treatment for reducing risk of developing a cardiac arrhythmia that include: (a) one or more of: performing an immunoassay to determine the level of CBG in a sample including blood, plasma, or serum obtained from a subject; performing an immunoassay to determine the level of GC in a sample including blood, plasma, or serum obtained from the subject; and performing an immunoassay to determine the level of NE in a sample including blood, plasma, or serum obtained from the subject; (b) comparing the determined level(s) of one or more of CBG, GC, and NE to reference level(s) of CBG, GC, and NE, respectively; and (c) selecting a subject having one or more of a determined level of CBG that is decreased as compared to a reference level of CBG, a determined level of GC that is elevated as compared to a reference level of GC, and a determined level of NE that is elevated as compared to a reference level of NE for administration of a treatment for reducing the risk of developing a cardiac arrhythmia. In some embodiments, step (a) includes determining the level of one of CBG, GC, and NE in the sample. In some embodiments, step (a) includes determining the level of two of CBG, GC, and NE in the sample. In some embodiments, step (a) includes determining the level of CBG, GC, and NE in the sample. In some embodiments, step (c) includes selecting a subject having two or three of a determined level of CBG that is decreased as compared to a reference level of CBG a determined level of GC that is elevated as compared to a reference level of GC, and a determined level of NE that is elevated as compared to a reference level of NE for administration of a treatment for reducing the risk of developing a cardiac arrhythmia. In some embodiments, step (c) includes selecting a subject having a determined level of CBG that is decreased as compared to a reference level of CBG, a determined level of GC that is elevated as compared to a reference level of GC, and a determined level of NE that is elevated as compared to a reference level of NE for administration of a treatment for reducing the risk of developing a cardiac arrhythmia. In some embodiments, the immunoassay used to determine the level of CBG is an enzyme-linked immunosorbent assay. In some embodiments, the immunoassay used to determine the level of GC is an enzyme-linked immunosorbent assay. In some embodiments, the immunoassay used to determine the level of NE is an enzyme-linked immunosorbent assay.

Also provided herein are methods of selecting a subject for administration of a treatment for reducing risk of developing a cardiac arrhythmia that include selecting a subject determined to have one or more of a level of CBG that is decreased as compared to a reference level of CBG, a level of GC that is elevated as compared to a reference level of GC, and a level of NE that is elevated as compared to a reference level of NE for administration of a treatment for reducing the risk of developing a cardiac arrhythmia. In some embodiments, such methods include selecting a subject determined to have two or three of a level of CBG that is decreased as compared to a reference level of CBG, a level of GC that is elevated as compared to a reference level of GC, and a level of NE that is elevated as compared to a reference level of NE for administration of a treatment for reducing the risk of developing a cardiac arrhythmia. In some embodiments, such methods include selecting a subject determined to have a level of CBG that is decreased as compared to a reference level of CBG, a level of GC that is elevated as compared to a reference level of GC, and a level of NE that is elevated as compared to a reference level of NE for administration of a treatment for reducing the risk of developing a cardiac arrhythmia.

Some embodiments of any of the methods described herein further include recording the selection of the subject for administration of the treatment in the subject's clinical record. In some embodiments, the subject's clinical record is a computer readable medium.

In some embodiments of any of the methods described herein, the treatment for reducing risk of developing a cardiac arrhythmia is selected from the group of: a SSRI, a NRI, a DRI, a SNRI, a NDRI, a TRI, an anti-clotting medication, a heart rate management medication, a heart rhythm management medication, a blood pressure management medication, a neutrophil elastase inhibitor, a cholesterol management medication, a cortisol management medication, and a sympathetic activity management medication. In some embodiments of any of the methods described herein, the treatment for reducing risk of developing a cardiac arrhythmia can include performing increased monitoring of the subject or performing a surgical procedure on the subject.

Also provided herein are methods of determining the efficacy of a treatment in a subject having or at increased risk of developing a cardiac arrhythmia that include: (a) one or more of: performing an immunoassay to determine the level of CBG in a first sample including blood, plasma, or serum obtained from a subject at a first time point; performing an immunoassay to determine the level of GC in a first sample including blood, plasma, or serum obtained from the subject at the first time point; and performing an immunoassay to determine the level of NE in a first sample including blood, plasma, or serum obtained from the subject at the first time point; (b) administering a treatment to the subject between the first time point and a second time point; (c) one or more of: performing an immunoassay to determine the level of CBG in a second sample including blood, plasma, or serum obtained from the subject at the second time point; performing an immunoassay to determine the level of GC in a second sample including blood, plasma, or serum obtained from the subject at the second time point; and performing an immunoassay to determine the level of NE in a second sample including blood, plasma, or serum obtained from the subject at the second time point; (c) comparing the determined level of one or more of CBG, GC, and NE at the second time point to the determined level of one or more of CBG, GC, and NE at the first time point; and (d) determining that the treatment administered to a subject having one or more of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point was not effective; or determining that the treatment administered to a subject having one or more of: a determined CBG level at the second time point that is about the same or increased as compared to a determined CBG level at the first time point; a determined GC level at the second time point that is about the same or decreased as compared to a determined GC level at the first time point; and a determined NE level at the second time point that is about the same or decreased as compared to a determined NE level at the first time point was effective. In some embodiments, step (d) includes determining that the treatment administered to a subject having one or more of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point was not effective. In some embodiments, step (a) includes determining the level of one of CBG, GC, and NE in the first sample, and step (c) includes determining the level of one of CBG, GC, and NE in the second sample. In some embodiments, step (a) includes determining the level of two of CBG, GC, and NE in the first sample, and step (c) includes determining the level of two of CBG, GC, and NE in the second sample. In some embodiments, step (a) includes determining the level of CBG, GC, and NE in the first sample, and step (c) includes determining the level of CBG, GC, and NE in the second sample.

In some embodiments, step (d) includes determining that the treatment administered to a subject having two or three of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point was not effective. In some embodiments, step (d) includes determining that the treatment administered to a subject having: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point was not effective. Some embodiments, where the treatment administered to the subject in step (b) is determined to be not effective further include administering to the subject an alternate treatment that is different from the treatment administered in step (b).

In some embodiments, step (d) includes determining that the treatment administered to a subject having one or more of: a determined CBG level at the second time point that is about the same or increased as compared to a determined CBG level at the first time point; a determined GC level at the second time point that is about the same or decreased as compared to a determined GC level at the first time point; and a determined NE level at the second time point that is about the same or decreased as compared to a determined NE level at the first time point was effective. In some embodiments, step (d) includes determining that the treatment administered to a subject having two or three of: a determined CBG level at the second time point that is about the same or increased as compared to a determined CBG level at the first time point; a determined GC level at the second time point that is about the same or decreased as compared to a determined GC level at the first time point; and a determined NE level at the second time point that is about the same or decreased as compared to a determined NE level at the first time point was effective. In some embodiments, step (d) includes determining that the treatment administered to a subject having: a determined CBG level at the second time point that is about the same or increased as compared to a determined CBG level at the first time point; a determined GC level at the second time point that is about the same or decreased as compared to a determined GC level at the first time point; and a determined NE level at the second time point that is about the same or decreased as compared to a determined NE level at the first time point was effective. Some embodiments where the treatment administered in step (b) to the subject is determined to be effective further include administering to the subject one or more doses of the treatment administered in step (b) to the subject.

Some embodiments of any of the methods described herein further include recording the determined efficacy of the treatment in a subject's clinical record. In some embodiments, the subject's clinical record is a computer readable medium.

In some embodiments of any of the methods described herein, the treatment administered in step (b) is a treatment for reducing risk of developing a cardiac arrhythmia. In some embodiments, the treatment for reducing the risk of developing a cardiac arrhythmia is selected from the group of: a SSRI, a NRI, a DRI, a SNRI, a NDRI, a TRI, an anti-clotting medication, a heart rate management medication, a heart rhythm management medication, a blood pressure management medication, a neutrophil elastase inhibitor, a cholesterol management medication, a cortisol management medication, and a sympathetic activity management medication. In some embodiments of any of the methods described herein, the treatment treatment for reducing risk of developing a cardiac arrhythmia can include performing increased monitoring of the subject or performing a surgical procedure on the subject.

Also provided herein are methods of monitoring a subject having or at increased risk of developing a cardiac arrhythmia that include: (a) one or more of: performing an immunoassay to determine the level of CBG in a first sample including blood, plasma, or serum obtained from a subject at a first time point; performing an immunoassay to determine the level of GC in a first sample including blood, plasma, or serum obtained from the subject at the first time point; and performing an immunoassay to determine the level of NE in a first sample including blood, plasma, or serum obtained from the subject at the first time point; (b) one or more of: performing an immunoassay to determine the level of CBG in a second sample including blood, plasma, or serum obtained from the subject at a second time point; performing an immunoassay to determine the level of GC in a second sample including blood, plasma, or serum obtained from a subject at the second time point; and performing an immunoassay to determine the level of NE in a second sample including blood, plasma, or serum obtained from a subject at the second time point; (c) comparing the determined level of one or more of CBG, GC, and NE at the second time point to the determined level of one or more of CBG, GC, and NE at the first time point; and (d) identifying a subject having one or more of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point as having a condition that is worsening; identifying a subject having one or more of: a determined CBG level at the second time point that is increased as compared to a determined CBG level at the first time point; a determined GC level at the lo second time point that is decreased as compared to a determined GC level at the first time point; and a determined NE level at the second time point that is decreased as compared to a determined NE level at the first time point as having a condition that is improving; or identifying a subject having one or more of: a determined CBG level at the second time point that is about the same as compared to a determined CBG level at the first time point; a determined GC level at the second time point that is about the same as compared to a determined GC level at the first time point; and a determined NE level at the second time point that is about the same as compared to a determined NE level at the first time point as having a condition that is static. In some embodiments, step (a) includes determining the level of one of CBG, GC, and NE in the first sample, and step (b) includes determining the level of one of CBG, GC, and NE in the second sample. In some embodiments, step (a) includes determining the level of two of CBG, GC, and NE in the first sample, and step (b) includes determining the level of two of CBG, GC, and NE in the second sample. In some embodiments, step (a) includes determining the level of CBG, GC, and NE in the first sample, and step (b) includes determining the level of CBG, GC, and NE in the second sample.

In some embodiments, step (d) includes identifying a subject having one or more of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point as having a condition that is worsening. In some embodiments, step (d) includes identifying a subject having two or three of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point as having a condition that is worsening. In some embodiments, step (d) includes identifying a subject having: a determined CBG level at the second time point that is reduced as compared to a determined

CBG level at the first time point; a determined GC level at the second time point that is elevated as compared to a determined GC level at the first time point; and a determined NE level at the second time point that is elevated as compared to a determined NE level at the first time point as having a condition that is worsening. In some embodiments, the subject is lo receiving a treatment, and the method further includes administering an alternate treatment that is different from the treatment that the subject has been receiving.

In some embodiments, step (d) includes identifying a subject having one or more of: a determined CBG level at the second time point that is increased as compared to a determined CBG level at the first time point; a determined GC level at the second time point that is decreased as compared to a determined GC level at the first time point; and a determined NE level at the second time point that is decreased as compared to a determined NE level at the first time point as having a condition that is improving. In some embodiments, step (d) includes identifying a subject having two or three of: a determined CBG level at the second time point that is increased as compared to a determined CBG level at the first time point; a determined GC level at the second time point that is decreased as compared to a determined

GC level at the first time point; and a determined NE level at the second time point that is decreased as compared to a determined NE level at the first time point as having a condition that is improving. In some embodiments, step (d) includes identifying a subject having: a determined CBG level at the second time point that is increased as compared to a determined CBG level at the first time point; a determined GC level at the second time point that is decreased as compared to a determined GC level at the first time point; and a determined NE level at the second time point that is decreased as compared to a determined NE level at the first time point as having a condition that is improving. In some embodiments, the subject is receiving a treatment, and the method further includes administering additional doses of the treatment to the subject.

In some embodiments, step (d) includes identifying a subject having one or more of: a determined CBG level at the second time point that is about the same as compared to a determined CBG level at the first time point; a determined GC level at the second time point that is about the same as compared to a determined GC level at the first time point; and a determined NE level at the second time point that is about the same as compared to a determined NE level at the first time point as having a condition that is static. In some embodiments, step (d) includes identifying a subject having two or three of: a determined CBG level at the second time point that is about the same as compared to a determined CBG level at the first time point; a determined GC level at the second time point that is about the same as compared to a determined GC level at the first time point; and a determined NE level at the second time point that is about the same as compared to a determined NE level at the first time point as having a condition that is static. In some embodiments, step (d) includes lo identifying a subject having: a determined CBG level at the second time point that is about the same as compared to a determined CBG level at the first time point; a determined GC level at the second time point that is about the same as compared to a determined GC level at the first time point; and a determined NE level at the second time point that is about the same as compared to a determined NE level at the first time point as having a condition that is static. In some embodiments, the subject is receiving a treatment, and the method further includes administering one or more additional doses of the treatment to the subject.

Some embodiments of any of the methods described herein further include recording the subject's determined condition in the subject's clinical record. In some embodiments, the subject's clinical record is a computer readable medium.

In some embodiments of any of the methods described herein, the immunoassay used to determine the level of CBG is an enzyme-linked immunosorbent assay, the immunoassay used to determine the level of GC is an enzyme-linked immunosorbent assay, and/or the immunoassay used to determine the level of NE is an enzyme-linked immunosorbent assay.

In some embodiments of any of the methods described herein, the subject is previously diagnosed as having cardiac arrhythmia and/or the subject is identified as having an increased risk of developing a cardiac arrhythmia. In some embodiments of any of the methods described herein, the subject does not have diabetes or metabolic syndrome, the subject has a BMI of ≤30, and/or the subject has a BMI of ≤25.

In some embodiments of any of the methods described herein, the subject does not have an elevated level of troponin C in a sample including blood, serum, or plasma obtained from the subject, as compared to a reference level of troponin C. In some embodiments, the reference level of troponin C is the level of troponin C present in a healthy subject or a population of healthy subjects, or a subject or a population of subjects determined to have a low risk of developing a cardiac arrhythmia.

In some embodiments of any of the methods described herein, the reference level of CBG is a level of CBG in a healthy subject or a population of healthy subjects, or a subject or a population of subjects determined to have a low risk of developing a cardiac arrhythmia;

the reference level of GC is a level of GC in a healthy subject or a population of healthy subjects, or in a subject or a population of subjects determined to have a low risk of developing a cardiac arrhythmia; and/or the reference level of NE is a level of NE in a healthy subject or a population of healthy subjects, or in a subject or a population of subjects lo determined to have a low risk of developing a cardiac arrhythmia.

Also provided herein are kits that include two or more of: (a) an antibody that binds specifically to CBG, (b) an antibody that binds specifically to GC; and (c) an antibody that binds specifically to NE.

Other features and advantages of the invention will be apparent from the following detailed description and figures, and from the claims.

### DETAILED DESCRIPTION

Provided herein are methods of determining a subject's risk of developing a cardiac arrhythmia, methods of treating a subject having or at risk of developing a cardiac arrhythmia, methods of selecting a treatment for a subject having or at risk of developing a cardiac arrhythmia, methods of selecting a subject for administration of a treatment for reducing risk of developing a cardiac arrhythmia, methods of determining the efficacy of a treatment in a subject having or at risk of developing a cardiac arrhythmia, and methods of monitoring a subject having or at increased risk of developing a cardiac arrhythmia based on a level of one or more of GC, CBG and NE in the subject. In some embodiments, the methods disclosed herein include performing an immunoassay to determine the levels of one or more of CBG, CG, and NE in the subject. Also provided are kits that include two or more of an antibody that binds specifically to GC, an antibody that binds specifically to GBC, and an antibody that binds specifically to NE. Non-limiting aspects of these methods and kits are described below. As can be appreciated in the art, the various aspects described below can be used in any combination without limitation.

As used herein, the word “a” before a noun represents one or more of the particular noun. For example, the phrase “a level” represents “one or more levels.”

As used herein, the term “about” means approximately, in the region of, roughly, or around. When used in conjunction with a numerical range, the term “about” modifies that range by extending the boundaries above and below the numerical values set forth. In general, the term “about” is used herein to modify a numerical value above and below the stated value by a variance of 10%.

As used herein, the term “BMI” means body mass index. BMI is calculated as body mass (e.g., measured in kilograms) divided by the square of the body height (e.g., measured in meters).

As used herein, the term “subject” means a vertebrate, including any member of the class mammalia, including humans, domestic and farm animals, and zoo, sports or pet animals, such as mouse, rabbit, pig, sheep, goat, cattle, horse (e.g., race horse), and higher primates. In some embodiments, the subject is a human.

As used herein, the term “treatment for reducing risk of developing cardiac arrhythmia” means a treatment that is administered to a subject in order to reduce the risk of developing a cardiac arrhythmia in a subject in need thereof. Non-limiting examples of treatments for reducing risk of developing cardiac arrhythmia include SSRI, a NRI, a DRI, a SNRI, a NDRI, a TRI, an anti-clotting medication, a heart rate management medication, a heart rhythm management medication, a blood pressure management medication, a neutrophil elastase inhibitor, a cholesterol management medication, a cortisol management medication, and a sympathetic activity management medication. In some examples, a treatment for reducing risk of developing cardiac arrhythmia can include performing increased monitoring of the subject or performing a surgical procedure on the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Cardiac Arrhythmias

The methods described herein relate generally to cardiac arrhythmias. Cardiac arrhythmias generically are a group of conditions in which a subject's heartbeat is abnormal. In some examples of the methods described herein, the cardiac arrhythmia can be a bradycardia (e.g., a resting heart rate below 60 beats per minute, below 55 beats per minute, below 50 beats per minute, below 45 beats per minute, or below 40 beats per minute) (e.g., sinus bradycardia, sinus arrest, AV block, or heart block).

In some examples of the methods described herein, the cardiac arrhythmia can be tachycardia (e.g., in subjects over 15 years of age, a resting heart rate of, e.g., greater than 95 beats per minute, greater than 100 beats per minute, greater than 105 beats per minute, greater than 110 beats per minute, greater than 115 beats per minute, greater than 120 beats per minute, greater than 125 beats per minute, greater than 130 beats per minute, or greater than 135 beats per minute) (e.g., sinus tachycardia).

In some examples of the methods described herein, the cardiac arrhythmia can be an atrial arrhythmia (e.g., sinus bradycardia, premature atrial contractions (PAC), atrial tachycardia, multifocal atrial tachycardia, supraventricular tachycardia (SVT), paroxysmal supraventricular tachycardia, atrial flutter, atrial fibrillation (Afib)). In some examples of the methods described herein, the cardiac arrhythmia is a junctional arrhythmia (e.g., AV nodal reentrant tachycardia, junctional rhythm, junctional tachycardia, and premature junctional contraction). In some examples of the methods described herein, the cardiac arrhythmia can be a ventricular arrhythmia (e.g., premature ventricular contractions (PVC), ventricular extra beats (VEB), accelerated idioventricular rhythm, ventricular tachycardia, monomorphic ventricular tachycardia, polymorphic ventricular tachycardia, ventricular fibrillation, and torsade de pointes (TdP). In some examples of the methods described herein, the cardiac arrhythmia is a heart block (e.g., a first degree heart block and a second degree heart block). In some examples of the methods described herein, the cardiac arrhythmia can be sudden arrhythmic death syndrome (SADS), neurocardiogenic syncope, and postural orthostatic tachycardia syndrome. In some examples of the methods described herein, the cardiac arrhythmia is caused by a heartbeat that occurs at irregular intervals.

Symptoms of a cardiac arrhythmias, when present, include, for example, palpitations, chest pain, lightheadedness, and shortness of breath. In more serious cases, symptoms can include fainting and cardiac arrest. Methods for diagnosing a cardiac arrhythmia are known in the art. For example, cardiac arrhythmias can be diagnosed using electrocardiogram, Holter monitor, echocardiogram, and exercise testing.

### Glucocorticoid

Natural glucocorticoids (GC), described as “glucocorticoid” or “GC” herein, are a class of naturally-produced corticosteroids that bind to the glucocorticoid receptor (GCR). A non-limiting example of a GC is cortisol. Methods of detecting a level of GC in a sample obtained from a subject (e.g., a sample comprising blood, serum or plasma) are known in the art. For example, a level of GC in a sample obtained from a subject (e.g., a sample lo comprising blood, serum, or plasma) can be determined using an immunoassay (e.g., an enzyme-linked immunosorbent assay). Examples of ELISAs that can be used to determine a level of a GC are commercially available from Antibodies Online (Atlanta, Ga.), Enzo Life Sciences, Inc. (Farmingdale, N.Y.), and Cayman Chemical (Ann Arbor, Mich.). As another example, a level of GC in a sample obtained from a subject can be determined by using any of a variety of spectrophotometric assays. As another example, a level of GC in a sample obtained from a subject can be determined by using any of a variety of chemical assays. Exemplary methods for determining a level of GC in a sample obtained from a subject are also described in Ziolkowska et al., Int. J. Mol. Med. 14:457-461, 2004; Ziolkowska et al., J. Steroid Biochem. Mol. Biol. 96:423-429, 2005; Chensue et al., Am. J. Pathol. 138:395-402, 1991; Gesquiere et al., Hormones and Behavior 54:410-416, 2008; Cristobal-Azkarate et al., Am. J. Primatol. 69:866-876, 2007; and Graham et al., General Comp. Endocrinol. 191:24-30, 2013.

### Corticosteroid-Binding Globulin

Corticosteroid-binding globulin (CBG) is a protein that transports glucocorticoids and progesterone in the blood and modulates the tissue availability of these hormones. CBG is a member of the serine protease inhibitor (SERPIN) family (see, e.g., Gardill et al., PLoS One 7(12):e52759, 2012; Lin et al., Mol. Cell Endrocrinol. 316:3-12, 2010; and Hammond et al., Proc. Natl. Acad. Sci. U.S.A. 84:5153-5157, 1987).

Examples of the amino acid sequence of precursor human CBG protein and mature human CBG protein are SEQ ID NO: 1 and SEQ ID NO: 2, respectively. An example of a cDNA sequence encoding human CBG protein is SEQ ID NO: 3.

Methods for determining a level of CBG are known in the art. For example, a level of CBG can be determined using an immunoassay (e.g., an ELISA). Examples of ELISAs that can be used to determine a level of CBG are commercially available from Elabscience (Bethesda, Md.), CusaBio (College Park, Md.), and GenWay Biotech Inc. (San Diego, Calif.). Additional exemplary methods that can be used to determine a level of CBG are described herein. As another example, a level of CBG in a sample obtained from a subject can be determined by using any of a variety of spectrophotometric assays. Non-limiting examples of assays that can be used to determine a level of CBG are described in, e.g., Westphal, Methods Enzymol. 15:761-796, 1969; Lapidus et al., Clin. Chem. 32:146-152, 1986; Coolens et al., J. Steroid Biochem. 26:197-202, 1987; Malisch et al., Gen. Comp. Endocrinol. 156:210-217, 2008; Brien, Clin. Endrocrinol. 14:193-212, 1981; Lewis et al., Clin. Chim. Acta 359:189-194, 2005; Fleshner et al., Endocrinol. 136:5336-5342, 1995; Lewis et al., Clin. Chim. Acta 328:121-128, 2003; Scott et al., J. Clin. Endocrinol. Metab. 71:639-644, 1990; Rosner et al., J. Clin. Endocrinol. Metab. 42:1064-1073, 1976; Schiller et al., J. Steroid Biochem. 7:55-59, 1976; and Rosner et al., Clin. Chem. 29:1389-1391, 1983.

Methods for determining a level of CBG mRNA are also known in the art. For examples, a level of CBG mRNA can be detected using, e.g., reverse-transcriptase (RT-PCR), real-time quantitative PCR, capillary-based quantitative PCR, fast qPCR, and TaqMan qPCR.

### Neutrophil Elastase

Neutrophil elastase (NE) is a serin proteinase in the same family as chymotrypsin. See, e.g., Takahashi et al., J. Biol. Chem. 263:14739-14747, 1988. Examples of the amino acid sequence of precursor human NE protein and mature human NE protein are SEQ ID NO: 4 and SEQ ID NO: 5, respectively. An example of a cDNA sequence encoding human NE protein is SEQ ID NO: 6.

Methods for determining a level of NE are known in the art. For example, a level of NE can be determined using an immunoassay (e.g., an ELISA). Examples of ELISAs that can be used to determine a level of NE are commercially available from Abcam (Cambridge, Mass.), Novus Biologicals (Littleton, Colo.), and R & D Systems (Minneapolis, Minn.). Additional exemplary methods that can be used to determine a level of NE are described herein. As another example, a level of NE in a sample obtained from a subject can be determined by using any of a variety of spectrophotometric or biochemical assays. Non-limiting examples of assays that can be used to determine a level of NE are described in, e.g., Redl et al., J. Infect. Dis. 164:383-388, 1991; Weitz et al., J. Clin. Invest. 78:155-162, 1986; Weitz et al., Ann. Intern. Med. 107:680-682, 1987; Buttle et al., Scandinavian J. Clin. Lab. Invest. 50:509-516, 1990; Sklar et al., J. Biol. Chem. 257:5471-5475, 1982; Sun et al., Lancet 5:182-190, 2004; and Grenda et al., Blood 100:3221-3228, 2002.

Methods for determining a level of NE mRNA are also known in the art. For examples, a level of NE mRNA can be detected using, e.g., reverse-transcriptase (RT-PCR), real-time quantitative PCR, capillary-based quantitative PCR, fast qPCR, and TaqMan qPCR.

Exemplary Methods for Determining a Level of CBG and/or NE in a Sample

Additional exemplary methods of determining the level of CBG and/or NE in a sample obtained from a subject (e.g., a sample including blood, serum, or plasma) are described below. In some embodiments, an immunoassay format is used to determine the level of one or both of CBG and NE in a sample. Any of a variety of immunoassay formats can be used in accordance with methods provided herein. In some embodiments, a CBG and/or NE level is determined by using an enzyme-linked immunosorbent assay (ELISA). In an ELISA, a protein (e.g., CBG and/or NE) is immobilized on a solid surface and bound by an antibody that is linked to an enzyme. The protein level is determined by measuring the activity of the conjugated enzyme on its substrate, e.g., by incubation with the substrate to produce a detectable reaction product. In some embodiments, an ELISA is a sandwich-type ELISA in which the protein to be detected and/or measured (e.g., CBG or NE) is immobilized on the solid surface by means of a second antibody specific to the protein (e.g., CBG or NE), wherein the second antibody is attached to the solid substrate (e.g., via adsorption, cross-linking, etc.). In some sandwich-type ELISA embodiments, a protein to be detected or measured (e.g., CBG or NE) is immobilized on a substrate having attached thereto a second antibody specific for the protein by incubating the substrate with a sample that contains the protein to be detected or measured. After incubation, the sample is washed under conditions for a time sufficient to remove non-bound protein. Exemplary enzymes that can be used in an ELISA include, without limitation, horseradish peroxidase, glucose oxidase, (3-galactosidase, and alkaline phosphatase. Enzymes used in ELISAs can be detected by determining or measuring the effect on the enzymes on their substrates. For example, a color change can be detected or measured when the enzyme acts on its substrate. In some ELISA embodiments, alkaline phosphatase acts on its substrate, p-nitrophenyl phosphate, to produce a reaction product that can be detected or measured at 405 nm. Similarly, β-galactosidase acts on its substrate, o-nitrophenyl-β-D-galactopyranoside (ONPG), to produce a reaction product that can be detected or measured at 410 nm. Fluorogenic substrates may also be used in ELISAs.

In some embodiments, a spectrophotometric assay is used to determine the level of one or both of CBG and NE in a sample. Any of a variety of spectrophotometric assays can be used in accordance with methods provided herein. In some embodiments, a CBG and/or NE level is determined by measuring the absorbance of CBG, NE, or both using a commercially-available spectrophotometer. A variety of commercially-available spectrophotometers are known in the art. In some embodiments, a single-beam spectrophotometer is used to determine the level of CBG, NE, or both in a sample. In some embodiments, a dual-beam spectrophotometer is used to determine the level of CBG, NE, or both in a sample. In general, the level of CBG and/or NE in a sample can be analyzed spectrophotometrically by placing the sample in a testing chamber (e.g., a cuvette), passing electromagnetic radiation of a given wavelength through the testing chamber, and measuring the intensity of electromagnetic radiation (e.g., visible light, infrared light, or ultraviolet radiation) that is transmitted through the testing chamber. By comparing the intensity of electromagnetic radiation that is transmitted through the testing chamber to the known initial intensity of electromagnetic radiation that is transmitted into the testing chamber, it is possible to determine the amount of electromagnetic radiation that was absorbed by a substance (e.g., CBG or NE) in the testing chamber. The amount of absorption can then be used to determine the amount of substance in the testing chamber. In some embodiments, the levels of CBG and NE are determined spectrophotometrically from the same sample. In some embodiments, the levels of CBG and NE are determined spectrophotometrically from different samples. Non-limiting spectrophotometric assays for determining a level of CBG or NE are described herein.

In some embodiments, a chemical assay is used to determine the level of one or both of CBG and NE in a sample. Any of a variety of chemical assays can be used in accordance with methods provided herein. In some embodiments, the levels of CBG and NE are determined using a chemical assay from the same sample. In some embodiments, the levels of CBG and NE are determined using a chemical assay from different samples. Non-limiting chemical assays that can be used to determine a level of CBG or NE are described herein.

In some embodiments, the level of CBG, NE, or both is determined using an automated or semi-automated analyzer. For example, any of the spectrophotometric, chemical, immunologic, or other methods for determining a level of CBG and/or NE described herein can be a component of an automated or semi-automated analyzer. Using an automated or semi-automated analyzer can increase the speed at which samples are analyzed, reduce the cost associated with analysis, reduce the amount of sample required for analysis, and increase the accuracy and consistency of the measured levels of CBG, NE, or both. A variety of automated and semi-automated analyzers are known, including, e.g., continuous flow analyzers, centrifugal analyzers, discrete auto analyzers, and spectrophotometric multi-well plate readers. Other suitable methods for determining the level of CBG and/or NE in a sample include, without limitation, antigen capture assays, quantitative Western blotting assays, radioimmunoassays (RIAs), flow cytometry assays, quantum dot assays, protein-based microarrays, and quantitative mass spectrometry methods such as, e.g., SELDI-TOF (surface-enhanced laser desorption/ionization- time of flight) mass spectrometry.

In some embodiments, the level of a CBG or NE is the level of a nucleic acid that encodes the CBG or NE, respectively, in the sample. Various methods of determining the level of a nucleic acid (e.g., an mRNA) encoding CBG or NE are known in the art and include, without limitation, Northern blot assays, quantitative RT-PCR assays, nuclease protection assays, microarray assays, and others. For example, CBG and NE mRNA levels can be measure using commercially available kits such as gene expression kits available from ThermoFisher Scientific (Waltham, Mass.). Additional methods for detecting CBG and NE levels are described herein.

Other non-liming methods of determining a level of CBG or NE include, e.g., measurement of CBG or NE activity. For example, NE activity can be determined using any of a variety of commercially available assays such as, without limitation, the Neutrophil Elastase Activity Assay Kit available from Cayman Chemical (Ann Arbor, Mich.). Other commercially available kits for measuring CBG or NE activity are known in the art. Non-limiting examples of assays for determining the level of CGB or NE activity are described in the references cited herein.

General guidance on these and other protein and nucleic acid detection methods can be found in, e.g., Green and Sambrook, Molecular Cloning: A Laboratory Manual 4th ed., Cold Spring Harbor Laboratory Press (Cold Spring Harbor, N.Y. 2012) and Current Protocols in Protein Science, John Wiley and Sons, Inc.: Hoboken, N.J.; Enna et al., eds. (2005), each of which is incorporated herein by reference in its entirety.

In some embodiments, methods provided herein include determining the level of one or more (e.g., 1, 2, or 3) of CBG, GC, or NE in a sample obtained from a subject (e.g., a sample including blood, plasma, or serum obtained from the subject). In some embodiments, methods provided herein include comparing the determined level of one or more (e.g., 1, 2, or 3) of the level of CBG, GC, or NE in a sample obtained from a subject to a reference level of CBG, GC, or NE, respectively (e.g., any of the reference levels of CBG, GC, and NE described herein).

In some embodiments, the level of one or more of CBG, GC, or NE in a sample obtained from a subject is determined to be elevated when compared to a reference level of lo CBG, GC, or NE, respectively (e.g., any of the reference levels of CBG, GC, and NE described herein). As used herein in reference to determined level of CBG, GC, or NE, the term “elevated” means at least a 0.5% increase (e.g., at least a 1% increase, at least a 2% increase, at least a 3% increase, at least a 4% increase, at least a 5% increase, at least a 6%, increase, at least a 7% increase, at least a 8% increase, at least a 9% increase, at least a 10% increase, at least a 12%, increase, at least a 14% increase, at least a 16% increase, at least a 18% increase, at least a 20% increase, at least a 22% increase, at least a 24% increase, at least a 26% increase, at least a 28% increase, at least a 30% increase, at least a 32% increase, at least a 34% increase, at least a 36% increase, at least a 38% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, or at least a 100% increase) in the determined level of CBG, GC, or NE as compared to the reference level of CBG, GC, or NE, respectively (e.g., any of the reference levels of CBG GC, and NE described herein).

In some embodiments, the level of one or more of CBG, GC, or NE in a sample obtained from a subject is determined to be decreased when compared to a reference level of CBG, GC, or NE, respectively (e.g., any of the reference levels of CBG, GC, and NE described herein). As used herein in reference to determined level of CBG, GC, or NE, the term “decreased” means at least a 0.5% decrease (e.g., at least a 1% decrease, at least a 2% decrease, at least a 3% decrease, at least a 4% decrease, at least a 5% decrease, at least a 6%, decrease, at least a 7% decrease, at least a 8% decrease, at least a 9% decrease, at least a 10% decrease, at least a 12%, decrease, at least a 14% decrease, at least a 16% decrease, at least a 18% decrease, at least a 20% decrease, at least a 22% decrease, at least a 24% decrease, at least a 26% decrease, at least a 28% decrease, at least a 30% decrease, at least a 32% decrease, at least a 34% decrease, at least a 36% decrease, at least a 38% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, or at least a 95% decrease) in the determined level of CBG, GC, or NE as compared to the reference level of CBG, GC, or NE, respectively (e.g., any of the reference levels of CBG, GC, and NE described herein).

In some embodiments, the level of one or more of CBG, GC, or NE in a sample obtained from a subject is determined to be about the same when compared to a reference level of CBG, GC, or NE, respectively (e.g., any of the reference levels of CBG, GC, and NE described herein). As used herein in reference to determined level of CBG, GC, or NE, the term “about the same” means less than a ±0.5% change in the determined level of CBG, GC, or NE as compared to the reference level of CBG, GC, or NE, respectively (e.g., any of the reference levels of CBG, GC, and NE described herein).

A reference level of CBG, GC, or NE can be, e.g., a level of CBG, GC, or NE, respectively, in a healthy subject or a population of healthy subjects. A reference level of CBG, GC, or NE can be, e.g., a level of CBG, GC, or NE, respectively, in a subject or a population of subjects determined to have a low risk of developing a cardiac arrhythmia. A reference level of CBG, GC, or NE can be, e.g., a level of CBG, GC, or NE, respectively, detected in the same subject at an earlier time point (e.g., 1 day earlier, 2 days earlier, 3 days earlier, 4 days earlier, 5 days earlier, 6 days earlier, 7 days earlier, 8 days earlier, 9 days earlier, 10 days earlier, 2 weeks earlier, 1 month earlier, 2 months earlier, 4 months earlier, 6 months earlier, 8 months earlier, 10 months earlier, 1 year earlier, 2 years earlier, 3 years earlier, 4 years earlier, 5 years earlier, 6 years earlier, 7 years earlier, 8 years earlier, 9 years earlier, or 10 years earlier).

### Point of Care Devices for Use in Determining a Subject's Risk of Developing a Cardiac Arrhythmia

In some embodiments, determining a subject's risk of developing a cardiac arrhythmia involves the use of a point of care device. For example, a point of care device can be used to collect, analyze, store, and/or transmit data (e.g., a subject's level of one or more of GC, CBG, and NE). Use of a point of care device when determining the level of one or more of GC, CBG and NE can facilitate the speed and/or accuracy of determining a subject's risk of developing a cardiac arrhythmia. In some embodiments, a point of care device is transportable, such that it is capable of being moved and used in a variety of locations (e.g., in locations where the testing is to take place).

In some embodiments, a point of care device is a stand-alone device. For example, a stand-alone point of care device can be used to determine the level of one or more of GC, CBG, and NE in a subject at a given location, and the determined level(s) can be identified at that location. In some embodiments, the level of one or more of GC, CBG and NE can be compared to a reference level of one or more of GC, CBG, and NE, respectively, to determine the subject's risk of developing a cardiac arrhythmia. For example, the stand-alone point of care device can determine the level of one or more of GC, CBG, and NE in the subject and in a separate reference or control sample at the same location, and the determined level of GC, CBG and NE in the subject can be compared to the determined reference or control level(s) to determine the subject's risk of developing a cardiac arrhythmia. Additionally and/or alternatively, the stand-alone point of care device can include a memory component in which is stored a reference or control level of one or more of GC, CBG, and NE, and the determined level of one or more of GC, CBG and NE in the subject can be compared to the stored reference level(s) to determine the subject's risk of developing a cardiac arrhythmia.

In some embodiments, a point of care device transmits and/or receives data from another device. For example, a point of care device can be used to determine the level of one or more of GC, CBG and NE in a subject at a given location, after which the point of care device can transmit the determined level(s) to another device for use in analyzing or comparing the determined levels to determine the subject's risk of developing a cardiac arrhythmia. As another example, a point of care device can be used to determine the level of one or more of GC, CBG, and NE in a subject at a given location, after which the point of care device receives data (e.g., reference or levels of one or more of GC, CBG and NE, respectively) from another device for use in analyzing or comparing the determined levels to determine the subject's risk of developing a cardiac arrhythmia. Data can be transmitted to or received from a point of care device in any of a variety of ways. For example, data can be transmitted or received wirelessly (e.g., via WiFi, cellular networks, radio, satellite, etc.). Additionally or alternatively, data can be transmitted or received over a physical connection (e.g., via hardwired telephone lines, ethernet cables, etc.)

In some embodiments, a point of care device includes a memory component that includes an algorithm for use in determining a subject's risk of developing a cardiac arrhythmia. For example, the level of one or more of GC, CBG and NE can be determined, and an algorithm included in the point of care device can be used to analyze the determined level(s) to determine the subject's risk of developing a cardiac arrhythmia. In some embodiments, a point of care device includes a memory component that includes more than one algorithm for use in determining a subject's risk of developing a cardiac arrhythmia. For example, different algorithms can be used to determine the risk of developing a cardiac arrhythmia in different subjects, depending on the subject's age, weight, sex, race, prior medical history, etc. In some embodiments, a point of care device transmits a determined level of one or more of GC, CBG and NE to a separate device that includes a memory component that includes one or more algorithms for use in determining a subject's risk of developing a cardiac arrhythmia.

In some embodiments, a point of care device can be used to inform the subject, the subject's family, the subject's primary care physician or attending physician, and/or the subject's insurance provider of the subject's determined risk of developing a cardiac arrhythmia.

As will be appreciated by those skilled in the art, a point of care device can also be used advantageously in accordance with any of the various other methods described herein, such as methods of treating a subject having or at risk of developing a cardiac arrhythmia, methods of selecting a treatment for a subject having or at risk of developing a cardiac arrhythmia, methods of selecting a subject for administration of a treatment for reducing risk of developing a cardiac arrhythmia, methods of determining the efficacy of a treatment in a subject having or at risk of developing a cardiac arrhythmia, and methods of monitoring a subject having or at increased risk of developing a cardiac arrhythmia.

### Treatment for Reducing Risk of Developing a Cardiac Arrhythmia

Non-limiting examples of treatments for reducing risk of developing a cardiac arrhythmia include, e.g., pharmacological agents that harmonize the hypothalamo-pituitary adrenal (HPA) axis, e.g., a selective serotonin reuptake inhibitor (S SRI), a norephinephrine reuptake inhibitor (NRI), a dopamine reuptake inhibitor (DRI), a serotonin/norephinephrine reuptake inhibitor (SNRT), a norepinephrine/dopamine reuptake inhibitor (NDRI), a triple reuptake inhibitor (TRI), an anti-clotting medication, a heart rate management medication, a heart rhythm management medication, a blood pressure management medication, a neutrophil elastase inhibitor, a cholesterol management medication, a cortisol management medication, and a sympathetic activity management medication. Additional examples of treatments for reducing risk of developing a cardiac arrhythmia include performing increased monitoring of the subject or performing a surgical procedure on the subject.

Non-limiting examples of SSRIs include, e.g., fluoxetine, fluvoxamine, escitalopram, paroxetine, fluoxetine, sertraline, citalopram, and paroxetine. Non-limiting examples of NRIs include, e.g., amedalin, CP-39,332, daledalin, edivoxetine, esreboxetine, lortalamine, nisoxetine, reboxetine, talopram, talsupram, tandamine, viloxazine, bupropion, ciclazindol, manifaxine, maprotiline, radafaxine, tapentadol, and teniloxazine. Non-limiting examples of DRIs include, e.g., altropane, amfonelic acid, amineptine, BTCP, DBL-583, difluoropine, GBR-12783, GBR-12935, GBR-13069, GBR-13098, GYKI-52895, iometopane, methylphenidate, RTI-229, vanoxerine, adrafinil, armodafinil, benztropine, bupropion, fluorenol, medifoxamine, metaphit, modafinil, rimcazole, and venlafaxine. Non-limiting examples of SNRIs include, e.g., venlafaxine, desvenlafaxine, duloxetine, milnacipran, levomilnacipran, sibutramine, and atomoxetine. Non-limiting examples of NDRIs include, e.g., amineptine, bupropion, desoxypipradrol, dexmethylphenidate, difemetorex, diphenylprolinol, ethylphenidate, fencamfamine, fencamine, lefetamine, methylenedioxypyrovalerone, methylphenidate, nomifensine, 0-2172, oxolinic acid, pipradrol, prolintane, pyrovalerone, tametraline, and WY-46824. Non-limiting examples of TRIs include, e.g., esketamine, ketamine, phencyclidine, tripelennamine, amitifadine, AN788, ansofaxine, centanafadine, dasotraline, Lu AA34893, Lu AA37096, NS-2360, tedatioxetine, and tesofensine.

Non-limiting examples of anti-clotting medications include anticoagulants (e.g., warfarin, dabigatran, rivaroxaban, edoxaban, and apixaban) and anti-platelet medications (e.g., aspirin, clopidogrel, dipyridamole, and ticlopidine). Additional examples of anti-clotting medications are known in the art.

Non-limiting examples of heart rate management medications include, beta blockers (e.g., atenolol, bisoprolol, carvedilol, metoprolol, nadolol, propranolol, timolol, and esmolol), calcium channel blockers (e.g., dilitiazem and verapamil), and current modulators (e.g., digoxin). Additional examples of heart rate management medications are known in the art.

Non-limiting examples of heart rhythm management medications include sodium channel blockers (e.g., flecainide, propafenone, quinidine, procainamide, disopyramide, lidocaine, phenytoin, and mexiletine), potassium channel blockers (e.g., amiodarone, sotalol, dofetilide, and ibutilide), and mixed sodium potassium channel (HCN) blockers (e.g., ivabradine). Additional examples of heart rhythm management medications are known in the art.

Non-limiting examples of blood pressure management medications include angiotensin converting enzyme (ACE) inhibitors (e.g., benazepril, captopril, enalapril, fosinopril, lisinopril, moexipril, perindopril, quinapril, ramipril, and trandolapril). Additional examples of blood pressure management medications are known in the art.

Non-limiting examples of neutrophil elastase management inhibitors include sivelestat, substituted benzoxazinones, and substituted azetidine-2, 4-diones. Additional examples of neutrophil elastase management inhibitors are known in the art.

Non-limiting examples of cholesterol management medications include statins (e.g., rosuvastatin, fluvastatin, atorvastatin, pitavastatin, lovastatin, pravastatin, and simvastatin). Additional examples of cholesterol management medications are known in the art.

Non-limiting examples of cortisol management medications include cortisol receptor antagonists (e.g., metyrapone), 11-beta-HSD type 1 inhibitors (e.g., corsolic acid, asiatic acid (CID 119034), Genins of Asiatic acid (CID 59752459), cabenxolone, and etomidate), and ACHT modulators (e.g., bromocriptine, cabergoline, pasireotide, and octreotide). Additional examples of cortisol management medications are known in the art.

Non-limiting examples of sympathetic activity management medications include anxiolytics (e.g., hydroxyzine (an antihistamine)), sleep treatments (e.g., continuous positive airway pressure (CPAP) therapy), and sleep medications (e.g., melatonin receptor agonists). Additional examples of sympathetic activity management medications are known in the art.

Non-limiting examples of performing a surgical procedure on a subject include ablation, Cox-Maze III, Cox-Maze IV, Wolf mini-maze, and implantation of a device (e.g., a defibrillator or a pacemaker). Additional examples of suitable surgical procedures that can be performed on a subject are known in the art.

Non-limiting examples of performing increased monitoring of a subject include, e.g., the performance of physical examinations, the performance of laboratory testing, the performance of electrocardiograms (ECGs), the use of a Holter monitor, the use of a cardiac event monitor, the performance of mobile cardiac telemetry, and/or the surgical implantation of an implantable cardiac monitoring device. Additional techniques for performing increased monitoring of a subject are known in the art.

Non-limiting examples of treatments for reducing risk of developing a cardiac arrhythmia include, without limitation, medications, surgical procedures, and electrical procedures.

Once identified as having or being at risk of developing a cardiac arrhythmia, the subject can be administered or instructed to self-administer one or more treatments for reducing risk of developing a cardiac arrhythmia (e.g., any of the treatments for reducing risk of developing a cardiac arrhythmia described herein or known in the art). Non-limiting examples of treatments for reducing risk of developing a cardiac arrhythmia include, without limitation, beta blockers, procainamide, amiodarone, dronedarone, dofetilide, ibutilide, propafenone, flecainide, anticoagulants (e.g., warfarin, heparin, dabigatran, rivaroxaban, edoxaban, apixaban), bloodthinners, and anti-platelet drugs (e.g., aspirin).

In some examples, one or more treatments for reducing risk of developing a cardiac arrhythmia can be administered to a subject once or multiple times over a period of time ranging from days to years. In some examples, one or more treatments for reducing risk of developing a cardiac arrhythmia can be formulated into a pharmaceutically acceptable composition for administration to a subject (e.g., a subject identified as having an increased risk of developing a cardiac arrhythmia using any of the methods described herein or a subject selected for administration of a treatment for reducing risk of developing a cardiac arrhythmia using any of the methods described herein). One or more treatments for reducing risk of developing a cardiac arrhythmia can be formulated together with one or more pharmaceutically acceptable carriers (additives) and/or diluents. One or more treatments for reducing risk of developing a cardiac arrhythmia can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used to formulate one or more treatments for reducing risk of developing a cardiac arrhythmia include, without limitation, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more treatments for reducing risk of developing a cardiac arrhythmia can be formulated for oral or parenteral (including subcutaneous, intramuscular, intravenous, and intradermal) administration. When formulated for oral administration, one or more treatments for reducing risk of developing a cardiac arrhythmia can be formulated in the form of a pill, tablet, or capsule. One or more treatments for reducing risk of developing a cardiac arrhythmia can be formulated as an aqueous and non-aqueous sterile injection solution that can contain, e.g., an anti-oxidant, a buffer, a bacteriostat, and a solute that renders the formulation isotonic with the blood of the intended recipient. One or more treatments for reducing risk of developing a cardiac arrhythmia can be provided in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, one or more treatments for reducing risk of developing a cardiac arrhythmia can be administered locally or systemically. In some cases, one or more treatments for reducing risk of developing a cardiac arrhythmia can be administered systemically, orally, or by injection to a subject (e.g., a human).

Effective doses of the treatment for reducing risk of developing a cardiac arrhythmia can vary depending on the level of the subject's determined risk of developing a cardiac arrhythmia, the route of administration, the age and general health condition of the subject, excipient usage, the possibility of co-usage with other therapeutic treatments, such as use of other agents, and the judgment of the treating physician.

An effective amount of a treatment for reducing risk of developing a cardiac arrhythmia can be any amount that delays the onset of a cardiac arrhythmia in a subject or reduces the risk of developing a cardiac arrhythmia in a subject (e.g., without producing significant toxicity to a subject).

If a subject fails to respond to a treatment for reducing risk of developing a cardiac arrhythmia, then the amount of the treatment for reducing risk of developing a cardiac arrhythmia can be increased (e.g., by two-fold, three-fold, five-fold, ten-fold, or more). After receiving this higher dose, the subject can be monitored for both responsiveness to the treatment for reducing risk of developing a cardiac arrhythmia and toxicity symptoms, and adjustments can be made accordingly. The effective amount can remain constant or can be adjusted as a sliding scale or variable dose depending on the subject's response to the treatment for reducing risk of developing a cardiac arrhythmia. Various factors can influence the actual effective amount used for a particular application. For example, the frequency of administration, duration of treatment, use of multiple treatment agents, route of administration, and level of the subject's risk of developing a cardiac arrhythmia may require an increase or decrease in the actual effective dose administered.

The frequency of administration of a treatment for reducing risk of developing a cardiac arrhythmia can be any amount that reduces the risk of developing a cardiac arrhythmia or delays the onset of a cardiac arrhythmia in the subject without producing significant toxicity. For example, the frequency of administration of a treatment for reducing risk of developing a cardiac arrhythmia can be from about two to about three times a week to about two to about three times a month. The frequency of administration of a treatment for reducing risk of developing a cardiac arrhythmia can remain constant or can be varied during the duration of treatment. A course of treatment with a treatment for reducing risk of developing a cardiac arrhythmia can include rest periods. For example, a composition containing one or more treatments for reducing risk of developing a cardiac arrhythmia can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatments for reducing risk of developing a cardiac arrhythmia, route of administration, and the level of the risk of developing a cardiac arrhythmia (e.g., determined using any of the methods described herein) may require an increase or decrease in administration frequency.

An effective duration for administering a composition containing one or more treatments for reducing risk of developing a cardiac arrhythmia can be any duration that reduces the risk of developing a cardiac arrhythmia or delays the onset of a cardiac arrhythmia in the subject (e.g., without producing significant toxicity in the subject). In some examples, the effective duration can vary from several days to several years, or longer.

Multiple factors can influence the actual effective duration used for a particular treatment for reducing risk of developing a cardiac arrhythmia. For example, an effective duration can vary with the frequency of administration, effective amount, use of multiple treatments for reducing risk of developing a cardiac arrhythmia, route of administration, and level of the subject's risk of developing a cardiac arrhythmia (e.g., as determined using any of the methods described herein).

In some embodiments, a treatment for reducing risk of developing a cardiac arrhythmia is a surgical procedure. Suitable examples of such surgical procedures include, without limitation, cardiac ablation (e.g., laser, heat, cold, or electrical), and implantation of a pacemaker. In some embodiments, a treatment for reducing risk of developing a cardiac arrhythmia is an electrical procedure (e.g., defibrillation or cardioversion).

In some embodiments, two or more treatments for reducing risk of developing a cardiac arrhythmia can be administered to a subject in combination. In some embodiments, two or more treatments for reducing risk of developing a cardiac arrhythmia can be administered to a subject simultaneously. In some embodiments, two or more treatments for reducing risk of developing a cardiac arrhythmia can be administered to a subject sequentially.

### Methods of Determining a Subject's Risk of Developing a Cardiac Arrhythmia

Provided herein are methods of determining a subject's risk of developing a cardiac arrhythmia (e.g., atrial fibrillation). In some cases, such methods include (a) one or more (e.g., 1, 2 or 3) of: determining the level of CBG in a sample obtained from a subject, determining the level of GC in a sample obtained from the subject, and determining the level of NE in a sample obtained from the subject, (b) comparing the determined level(s) of one or more (e.g., 1, 2, or 3) of CBG, GC, and NE to reference level(s) of CBG, GC, and NE, respectively, and (c) identifying a subject having one or more (e.g., 1, 2, or 3) of a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia (e.g., as compared to a subject determined to not have any of a determined level of CBG that is decreased as compared to a reference level of CBG a determined level of GC that is elevated as compared to a reference level of GC, or a determined level of NE that is elevated as compared to a reference level of NE); or identifying a subject having one or more (e.g., 1, 2, or 3) of a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia (e.g., as compared to a subject determined to not have any of a determined level of CBG that is about the same or elevated as compared to a reference level of CBG a determined level of GC that is about the same or decreased as compared to a reference level of GC, or a determined level of NE that is about the same or decreased as compared to a reference level of NE). In some embodiments, step (a) includes performing an assay (e.g., an immunoassay) to determine the level(s) of one or more (e.g., 1, 2, or 3) of CBG, GC, and/or NE in a sample. Other exemplary assays for determining the levels of one or more (e.g., 1, 2, or 3) of CBG, GC, and/or NE in a sample are disclosed herein. In some embodiments, step (a) includes determining the level(s) of one or more (e.g., 1, 2, or 3) of CBG, GC, and/or NE in a sample, wherein the sample includes blood, plasma, or serum obtained from the subject.

In some embodiments, the methods of determining a subject's risk of developing a cardiac arrhythmia include identifying a subject determined to have one or more (e.g., 1, 2 or 3) of a level of CBG that is decreased as compared to a reference level of CBG a level of GC that is elevated as compared to a reference level of GC, and a level of NE that is elevated as compared to a reference level of NE as having an increased risk of developing a cardiac arrhythmia (e.g., as compared to a subject determined to not have any of a level of CBG that is decreased as compared to a reference level of CBG a level of GC that is elevated as compared to a reference level of GC, or a level of NE that is elevated as compared to a reference level of NE). In some embodiments, methods of determining a subject's risk of developing a cardiac arrhythmia include identifying a subject determined to have one or more (e.g., 1, 2 or 3) of a level of CBG that is about the same or elevated as compared to a reference level of CBG a level of GC that is about the same or decreased as compared to a reference level of GC, and a level of NE that is about the same or decreased as compared to a reference level of NE as having a decreased risk of developing a cardiac arrhythmia (e.g., as compared to a subject determined to not have any of a level of CBG that is about the same or elevated as compared to a reference level of CBG, a level of GC that is about the same or decreased as compared to a reference level of GC, or a level of NE that is about the same or decreased as compared to a reference level of NE).

In some embodiments, methods of determining a subject's risk of developing a cardiac arrhythmia include determining the level of one of CBG, GC, or NE in a sample, and comparing the determined level of CBG, GC, or NE to a reference level of CBG, GC, or NE, respectively. In some embodiments, methods of determining a subject's risk of developing a cardiac arrhythmia include determining the levels of two of CBG, GC, or NE in a sample (e.g., CBG and GC, CPB and NE, or GC and NE), and comparing the determined levels of CBG, GC, or NE to reference levels of CBG, GC, or NE, respectively. In some embodiments, methods of determining a subject's risk of developing a cardiac arrhythmia include determining the levels of each of CBG, GC, and NE in a sample, and comparing the determined levels of CBG, GC, and NE to reference levels of CBG, GC, or NE, respectively.

In some embodiments, a subject is determined to have an increased risk of developing a cardiac arrhythmia prior to observing or detecting conventional symptoms or other predictive indicators of cardiac arrhythmia. For example, a subject can be determined to have an increased risk of developing a cardiac arrhythmia at a time point when the subject does not present with any other traditional risk factors for cardiac arrhythmia, such as diabetes, obesity, or metabolic syndrome. In some examples, a subject can be determined to have an increased risk of developing a cardiac arrhythmia when the subject has a BMI of about 30 or less (e.g., about 29 or less, about 28 or less, about 27 or less, about 26 or less, about 25 or less). In some examples, a subject can be determined to have an increased risk of developing a cardiac arrhythmia when the subject does not have an elevated level of troponin C in a sample (e.g., a sample including blood, serum, or plasma) obtained from the subject, as compared to a reference level of troponin C.

In any of the methods disclosed herein, a reference level of CBG, CG, NE, and/or troponin C can be, e.g., a level(s) in a subject not presenting with one or more symptoms of cardiac arrhythmia and/or not diagnosed as having cardiac arrhythmia, a level(s) in a healthy subject or a population of healthy subjects, a threshold level(s), a level(s) in a subject or population of subjects not identified as having a genetic risk (e.g., an elevated genetic risk) of developing cardiac arrhythmia, a level(s) in a sample from a subject or a population of subjects that has/have no history of a cardiac arrhythmia, or a level(s) in a sample from a subject or a population of subjects that do not have a genetically-related family member diagnosed or identified as having a cardiac arrhythmia.

In some embodiments, after a subject is determined to have an increased risk of developing a cardiac arrhythmia, the subject is monitored for the development of symptoms associated with a cardiac arrhythmia. In some embodiments, after a subject is determined to have an increased risk of developing a cardiac arrhythmia, the subject's risk of developing a cardiac arrhythmia is determined at periodic intervals, e.g., every 1, 2, 3, 4, 5, or 6 days, every 1, 2, 3, or 4 weeks, every 1, 2, 3, 4, 5, 6, 7, 8, 9, 10, or 11 months, or every 1, 2, 3, 4, 5, 6, 7, 8, 9, 10, or more years. In some embodiments, the subject's risk of developing a cardiac arrhythmia is determined at equal periodic intervals. In some embodiments, the subject's risk of developing a cardiac arrhythmia is determined at irregular intervals.

After a subject has been determined to have an increased risk of developing a cardiac arrhythmia, the subject can be treated with one or more (e.g., 1, 2, 3, 4, 5, 6, 7, 8, 9, 10, or more) of the treatments for reducing risk of developing a cardiac arrhythmia described herein. In some embodiments, when a subject is treated with two or more treatments for reducing risk of developing a cardiac arrhythmia, the two or more treatments for reducing risk of developing a cardiac arrhythmia are administered simultaneously to the subject. In some embodiments, when a subject is treated with two or more treatments for reducing risk of developing a cardiac arrhythmia, the two or more treatments for reducing risk of developing a cardiac arrhythmia are administered sequentially to the subject. In some embodiments, after a subject has been determined to have an increased risk of developing a cardiac arrhythmia, and after a treatment for reducing risk of developing a cardiac arrhythmia is administered, the efficacy of the treatment can be determined by one or more of the methods disclosed herein.

Some embodiments further include recording a subject's risk of developing a cardiac arrhythmia in the subject's medical record (e.g., a computer readable medium). Some examples further include informing the subject, the subject's family, and/or the subject's primary care physician or attending physician of the subject's determined risk of developing a cardiac arrhythmia. Some examples further include informing the subject's insurance provider of the subject's risk of developing a cardiac arrhythmia.

### Methods of Treating a Subject Having or at Risk of Developing a Cardiac Arrhythmia

Also provided herein are methods of treating a subject. Some embodiments of these methods include: (a) one or more (e.g., 1, 2 or 3) of: determining the level of CBG in a sample obtained from a subject, determining the level of GC in a sample obtained from the subject, and determining the level of NE in a sample obtained from the subject; (b) comparing the determined level(s) of one or more of CBG, GC, and NE to reference level(s) of CBG, GC, and NE, respectively; (c) selecting a subject having one or more (e.g., 1, 2, or 3) of a determined level of CBG that is decreased as compared to a reference level of CBG a determined level of GC that is elevated as compared to a reference level of GC, and a determined level of NE that is elevated as compared to a reference level of NE; and (d) administering a treatment for reducing risk of developing a cardiac arrhythmia (e.g., any of the treatments for reducing risk of developing a cardiac arrhythmia described herein or known in the art) to the selected subject. In some embodiments, step (a) includes performing an assay (e.g., an immunoassay) to determine the level(s) of one or more (e.g., 1, 2, or 3) of CBG, GC, and/or NE in a sample. Additional assays that can be used to determine the level(s) of one or more (e.g., 1, 2, or 3) of CBG, GC, and/or NE in a sample are disclosed herein. In some embodiments, step (a) includes determining the level(s) of one or more (e.g., 1, 2, or 3) of CBG, GC, and/or NE in a sample, where the sample includes blood, plasma, or serum obtained from the subject.

Some embodiments of the methods of treating a subject include selectively administering to a subject determined to have one or more (e.g., 1, 2 or 3) of a level of CBG that is decreased as compared to a reference level of CBG a level of GC that is elevated as compared to a reference level of GC, and a level of NE that is elevated as compared to a reference level of NE, a therapeutically effective dose of a treatment for reducing the risk of a cardiac arrhythmia (e.g., any of the treatments for reducing the risk of a cardiac arrhythmia described herein or known in the art).

In some embodiments, the methods of treating a subject provided herein include determining the level of one of CBG, GC, or NE in a sample, and comparing the determined level of CBG, GC, or NE to a reference level of CBG, GC, or NE, respectively. In some embodiments, the methods of treating a subject provided herein include determining the levels of two of CBG, GC, or NE in a sample (e.g., CBG and GC, CBG and NE, or GC and NE), and comparing the determined levels of CBG, GC, or NE to reference levels of CBG, GC, or NE, respectively. In some embodiments, methods of treating a subject provided herein include determining the levels of each of CBG, GC, and NE in a sample, and comparing the determined levels of CBG, GC, and NE to reference levels of CBG, GC, or NE, respectively.

In some embodiments, the administering of a therapeutically effective dose of a treatment for reducing the risk of a cardiac arrhythmia to the subject results in a reduction in the risk of developing a cardiac arrhythmia in the subject or delays the onset of a cardiac arrhythmia in the subject. In some embodiments, the administering of a therapeutically effective dose of a treatment for reducing the risk of a cardiac arrhythmia is performed prior to observing or detecting other symptoms or other predictive indicators of cardiac arrhythmia in the subject. For example, the methods of treatment provided herein can be performed when the subject does not clinically present with traditional risk factors such as diabetes, obesity, or metabolic syndrome. In some examples, the methods of treatment are performed when the subject has a BMI of about 30 or less (e.g., about 29 or less, about 28 or less, about 27 or less, about 26 or less, about 25 or less). In some examples, the methods of treatment are performed when the subject does not have an elevated level of troponin C in a sample (e.g., a sample including blood, serum, or plasma) obtained from the subject, as compared to a reference level of troponin C.

In any of the methods disclosed herein, a reference level of CBCs CG, NE, and/or troponin C can be, e.g., a level(s) in a subject not presenting with one or more symptoms of cardiac arrhythmia and/or not diagnosed as having cardiac arrhythmia, a level(s) in a healthy subject or a population of healthy subjects, a threshold level(s), a level(s) in a subject or population of subjects not identified as having a genetic risk (e.g., an elevated genetic risk) of developing cardiac arrhythmia, a level(s) in a sample from a subject or a population of subjects that has/have no history of a cardiac arrhythmia, or a level(s) in a sample from a subject or a population of subjects that do not have a genetically-related family member diagnosed or identified as having a cardiac arrhythmia.

In some examples of any of the methods of treatment described herein, the subject is administered or selectively administered a therapeutically effective amount of any one or more (e.g., 1, 2, 3, 4, 5, 6, 7, 8, 9, 10, or more) of the treatments for reducing risk of developing a cardiac arrhythmia disclosed herein or known in the art. In some embodiments, when the subject is administered or selectively administered two or more (e.g., 1, 2, 3, 4, 5, 6, 7, 8, 9, 10, or more) treatments for reducing risk of developing a cardiac arrhythmia, the two or more treatments for reducing risk of developing a cardiac arrhythmia are administered to the subject in combination. In some embodiments, when the subject is administered or selectively administered two or more (e.g., 1, 2, 3, 4, 5, 6, 7, 8, 9, 10, or more) treatments for reducing risk of developing a cardiac arrhythmia, the two or more treatments for reducing risk of developing a cardiac arrhythmia are sequentially administered to the subject.

In some embodiments, after a subject is administered or selectively administered a treatment for reducing risk of developing a cardiac arrhythmia, the subject is further monitored for the development of symptoms associated with a cardiac arrhythmia. In some embodiments, the subject's risk of developing a cardiac arrhythmia is further determined after administering or selectively administering a treatment for reducing the risk of developing a cardiac arrhythmia to the subject. For example, after a subject is administered or selectively administered a treatment for reducing the risk of developing a cardiac arrhythmia, the subject's risk of developing a cardiac arrhythmia is determined at periodic intervals, e.g., every 1, 2, 3, 4, 5, or 6 days, every 1, 2, 3, or 4 weeks, every 1, 2, 3, 4, 5, 6, 7, 8, 9, 10, or 11 months, or every 1, 2, 3, 4, 5, 6, 7, 8, 9, 10, or more years. In some embodiments, the subject's risk of developing a cardiac arrhythmia is determined at equal periodic intervals after administering or selectively administering a treatment for reducing risk of developing a cardiac arrhythmia to the subject. In some embodiments, the subject's risk of developing a cardiac arrhythmia is determined at irregular intervals after administering or selectively administering a treatment for reducing risk of developing a cardiac arrhythmia to the subject.

Some embodiments further include recording in the subject's medical record (e.g., a computer readable medium) that the subject should be administered or selectively administered a treatment for reducing risk of developing a cardiac arrhythmia or that the subject has been administered or selectively administered a treatment for reducing risk of developing a cardiac arrhythmia. Some examples further include informing the subject, the subject's family, and/or the subject's primary care physician or attending physician that the subject should be administered or selectively administered a treatment for reducing risk of developing a cardiac arrhythmia or that the subject has been administered or selectively administered a treatment for reducing risk of developing a cardiac arrhythmia. Some examples further include informing the subject's insurance provider that the subject should be administered or selectively administered a treatment for reducing risk of developing a cardiac arrhythmia or that the subject has been administered or selectively administered a treatment for reducing risk of developing a cardiac arrhythmia.

### Methods of Selecting a Treatment for a Subject Having or at Risk of Developing a Cardiac Arrhythmia

Also provided herein are methods of selecting a treatment for a subject. In some embodiments, these methods include: (a) one or more (e.g., 1, 2, or 3) of: determining the level of CBG in a sample including blood, plasma, or serum obtained from a subject;

determining the level of GC in a sample including blood, plasma, or serum obtained from the subject, and determining the level of NE in a sample including blood, plasma, or serum obtained from the subject; (b) comparing the determined level(s) of one or more (e.g., 1, 2, or 3) of CBG, GC, and NE to reference level(s) of CBG, GC, and NE, respectively; and (c) selecting a treatment for reducing risk of developing a cardiac arrhythmia (e.g., any of the treatments for reducing risk of developing a cardiac arrhythmia described herein or known in the art) for a subject having one or more (e.g., 1, 2, or 3) of a determined level of CBG that is decreased as compared to a reference level of CBG a determined level of GC that is elevated as compared to a reference level of GC, and a determined level of NE that is elevated as compared to a reference level of NE. In some embodiments, step (a) includes performing an assay (e.g., an immunoassay) to determine the level(s) of one or more of CBG, GC, and/or NE in a sample. Additional exemplary assays for determining the level(s) of one or more (e.g., 1, 2, or 3) of CBG, GC, and/or NE in a sample are disclosed herein. In some embodiments, step (a) includes determining the level(s) of one or more (e.g., 1, 2, or 3) of CBG, GC, and/or NE in a sample, where the sample includes blood, plasma, or serum obtained from the subject.

In some embodiments, the methods of selecting a treatment for reducing risk of developing a cardiac arrhythmia for a subject include selecting a treatment for reducing risk of developing a cardiac arrhythmia for a subject determined to have one or more (e.g., 1, 2, or 3) of a level of CBG that is decreased as compared to a reference level of CBG a level of GC that is elevated as compared to a reference level of GC, and a level of NE that is elevated as compared to a reference level of NE.

In some embodiments, the methods of selecting a treatment for reducing risk of developing a cardiac arrhythmia for a subject include determining the level of one of CBG GC, or NE in a sample, and comparing the determined level of CBG, GC, or NE to a reference level of CBG, GC, or NE, respectively. In some embodiments, the methods of selecting a treatment for reducing risk of developing a cardiac arrhythmia for a subject include determining the levels of two of CBG, GC, or NE in a sample (e.g., CBG and GC, CPB and NE, or GC and NE), and comparing the determined levels of CBG, GC, or NE to reference levels of CBG, GC, or NE, respectively. In some embodiments, the methods of selecting a treatment for reducing risk of developing a cardiac arrhythmia for a subject include determining the levels of each of CBG, GC, and NE in a sample, and comparing the determined levels of CBG, GC, and NE to reference levels of CBG, GC, or NE, respectively.

In some embodiments, the methods of selecting a treatment for reducing risk of developing a cardiac arrhythmia for a subject is performed prior to observing or detecting other symptoms or other predictive indicators of cardiac arrhythmia in the subject. For example, the methods of selecting a treatment for reducing risk of developing a cardiac arrhythmia for a subject can be performed when the subject does not clinically present with traditional risk factors such as diabetes, obesity, or metabolic syndrome. In some examples, the methods of selecting a treatment for reducing risk of developing a cardiac arrhythmia for a subject is performed when the subject has a BMI of about 30 or less (e.g., about 29 or less, about 28 or less, about 27 or less, about 26 or less, about 25 or less). In some examples, the methods of selecting a treatment for reducing risk of developing a cardiac arrhythmia for a subject is performed when the subject does not have an elevated level of troponin C in a sample (e.g., a sample including blood, serum, or plasma) obtained from the subject, as compared to a reference level of troponin C.

In any of the methods disclosed herein, a reference level of CBCs CG, NE, and/or troponin C can be, e.g., a level(s) in a subject not presenting with one or more symptoms of cardiac arrhythmia and/or not diagnosed as having cardiac arrhythmia, a level(s) in a healthy subject or a population of healthy subjects, a threshold level(s), a level(s) in a subject or population of subjects not identified as having a genetic risk (e.g., an elevated genetic risk) of developing cardiac arrhythmia, a level(s) in a sample from a subject or a population of subjects that has/have no history of a cardiac arrhythmia, or a level(s) in a sample from a subject or a population of subjects that do not have a genetically-related family member diagnosed or identified as having a cardiac arrhythmia.

In some examples of any of the methods of treatment described herein, the selected treatment is any one or more (e.g., 1, 2, 3, 4, 5, 6, 7, 8, 9, 10, or more) of the treatments for reducing risk of developing a cardiac arrhythmia disclosed herein or known in the art. Some embodiments of the methods of selecting a treatment for a subject further include administering a selected treatment for reducing risk of developing a cardiac arrhythmia to the subject. In some embodiments, when the subject is administered two or more (e.g., 1, 2, 3, 4, 5, 6, 7, 8, 9, 10, or more) treatments for reducing risk of developing a cardiac arrhythmia, the two or more treatments for reducing risk of developing a cardiac arrhythmia are administered to the subject in combination. In some embodiments, when the subject is administered two or more (e.g., 1, 2, 3, 4, 5, 6, 7, 8, 9, 10, or more) treatments for reducing risk of developing a cardiac arrhythmia, the two or more treatments for reducing risk of developing a cardiac arrhythmia are sequentially administered to the subject. In some embodiments, after a treatment for reducing risk of developing a cardiac arrhythmia is selected for the subject and the selected treatment for reducing risk of developing a cardiac arrhythmia is administered to the subject, the subject can be monitored for the development of symptoms associated with a cardiac arrhythmia. For example, after a treatment for reducing risk of developing a cardiac arrhythmia is selected for the subject and the selected treatment for reducing risk of developing a cardiac arrhythmia is administered to the subject, the subject can be monitored at periodic intervals, e.g., every 1, 2, 3, 4, 5, or 6 days, every 1, 2, 3, or 4 weeks, every 1, 2, 3, 4, 5, 6, 7, 8, 9, 10, or 11 months, or every 1, 2, 3, 4, 5, 6, 7, 8, 9, 10, or more years. For example, after a treatment for reducing risk of developing a cardiac arrhythmia is selected for the subject and the selected treatment for reducing risk of developing a cardiac arrhythmia is administered to the subject, the subject can be monitored at irregular intervals. Some embodiments further include recording the selected treatment for reducing risk of developing a cardiac arrhythmia for the subject in the subject's medical record (e.g., a computer readable medium). Some examples further include informing the subject, the subject's family, and/or the subject's primary care physician or attending physician of the selected treatment for reducing risk of developing a cardiac arrhythmia for the subject. Some examples further include informing the subject's insurance provider of the selected treatment for reducing risk of developing a cardiac arrhythmia for the subject.

### Methods of Selecting a Subject for Administration of a Treatment for Reducing Risk of Developing a Cardiac Arrhythmia

Also provided herein are methods of selecting a subject for administration of a treatment for reducing risk of developing a cardiac arrhythmia (e.g., any of the treatments for reducing risk of developing a cardiac arrhythmia described herein or known in the art). In some embodiments, such methods include: (a) one or more (e.g., 1, 2, or 3) of: determining the level of CBG in a sample obtained from a subject, determining the level of GC in a sample obtained from the subject, and determining the level of NE in a sample obtained from the subject; (b) comparing the determined level(s) of one or more (e.g., 1, 2, or 3) of CBG GC, and NE to reference level(s) of CBG, GC, and NE, respectively; and (c) selecting a subject having one or more (e.g., 1, 2, or 3) of a determined level of CBG that is decreased as compared to a reference level of CBG a determined level of GC that is elevated as compared to a reference level of GC, and a determined level of NE that is elevated as compared to a reference level of NE for administration of a treatment for reducing the risk of developing a cardiac arrhythmia (e.g., any of the treatments for reducing risk of developing a cardiac arrhythmia described herein or known in the art). In some embodiments, step (a) includes performing an assay (e.g., an immunoassay) to determine the level(s) of one or more (e.g., 1, 2, or 3) of CBG, GC, and/or NE in a sample. Additional exemplary assays for determining the levels of one or more (e.g., 1, 2, or 3) of CBG, GC, and/or NE in a sample are described herein. In some embodiments, step (a) includes determining the level(s) of one or more (e.g., 1, 2, or 3) of CBG, GC, and/or NE in a sample, where the sample includes blood, plasma, or serum obtained from the subject.

In some embodiments, methods of selecting a subject for administration of a treatment for reducing risk of developing a cardiac arrhythmia include selecting a subject determined to have one or more (e.g., 1, 2, or 3) of a level of CBG that is decreased as compared to a reference level of CBG a level of GC that is elevated as compared to a reference level of GC, and a level of NE that is elevated as compared to a reference level of NE for administration of a treatment for reducing risk of developing a cardiac arrhythmia (e.g., any of the treatments for reducing risk of developing a cardiac arrhythmia described herein or known in the art).

In some embodiments, the methods of selecting a subject for administration of a treatment for reducing risk of developing a cardiac arrhythmia include determining the level of one of CBG, GC, or NE in a sample, and comparing the determined level of CBG, GC, or NE to a reference level of CBG, GC, or NE, respectively. In some embodiments, the methods of selecting a subject for administration of a treatment for reducing risk of developing a cardiac arrhythmia include determining the levels of two of CBG, GC, or NE in a sample (e.g., CBG and GC, CPB and NE, or GC and NE), and comparing the determined levels of CBG, GC, or NE to reference levels of CBG, GC, or NE, respectively. In some embodiments, the methods of selecting a subject for administration of a treatment for reducing risk of developing a cardiac arrhythmia include determining the levels of each of CBG, GC, and NE in a sample, and comparing the determined levels of CBG, GC, and NE to reference levels of CBG, GC, or NE, respectively.

In some embodiments, the methods of selecting a subject for administration of a treatment for reducing risk of developing a cardiac arrhythmia is performed prior to observing or detecting other symptoms or other predictive indicators of cardiac arrhythmia in the subject. For example, the methods of selecting a subject for administration of a treatment for reducing risk of developing a cardiac arrhythmia can be performed when the subject does not clinically present with traditional risk factors such as diabetes, obesity, or metabolic syndrome. In some examples, the methods of selecting a subject for administration of a treatment for reducing risk of developing a cardiac arrhythmia is performed when the subject has a BMI of about 30 or less (e.g., about 29 or less, about 28 or less, about 27 or less, about 26 or less, about 25 or less). In some examples, the methods of selecting a subject for administration of a treatment for reducing risk of developing a cardiac arrhythmia is performed when the subject does not have an elevated level of troponin C in a sample (e.g., a sample including blood, serum, or plasma) obtained from the subject, as compared to a reference level of troponin C.

In any of the methods disclosed herein, a reference level of CBCs CG, NE, and/or troponin C can be, e.g., a level(s) in a subject not presenting with one or more symptoms of cardiac arrhythmia and/or not diagnosed as having cardiac arrhythmia, a level(s) in a healthy subject or a population of healthy subjects, a threshold level(s), a level(s) in a subject or population of subjects not identified as having a genetic risk (e.g., an elevated genetic risk) of developing cardiac arrhythmia, a level(s) in a sample from a subject or a population of subjects that has/have no history of a cardiac arrhythmia, or a level(s) in a sample from a subject or a population of subjects that do not have a genetically-related family member diagnosed or identified as having a cardiac arrhythmia.

A subject can be selected for administration of any one or more (e.g., 1, 2, 3, 4, 5, 6, 7, 8, 9, 10, or more) of the treatments for reducing risk of developing a cardiac arrhythmia disclosed herein or known in the art. In some embodiments, after a subject is selected for administration of a treatment for reducing risk of developing a cardiac arrhythmia, the subject is administered one or more treatments for reducing risk of developing a cardiac arrhythmia. In some embodiments, when the subject is administered two or more (e.g., 1, 2, 3, 4, 5, 6, 7, 8, 9, 10, or more) treatments for reducing risk of developing a cardiac arrhythmia, the two or more treatments for reducing risk of developing a cardiac arrhythmia are administered to the subject in combination. In some embodiments, when the subject is administered two or more (e.g., 1, 2, 3, 4, 5, 6, 7, 8, 9, 10, or more) treatments for reducing risk of developing a cardiac arrhythmia, the two or more treatments for reducing risk of developing a cardiac arrhythmia are sequentially administered to the subject. In some embodiments, after a subject is selected for administration of a treatment for reducing risk of developing a cardiac arrhythmia and the subject is administered a treatment for reducing risk of developing a cardiac arrhythmia, the subject can be monitored for the development of symptoms associated with a cardiac arrhythmia. For example, after a subject is selected for administration of a treatment for reducing risk of developing a cardiac arrhythmia and the subject is administered a treatment for reducing risk of developing a cardiac arrhythmia, the subject can be monitored at periodic intervals, e.g., every 1, 2, 3, 4, 5, or 6 days, every 1, 2, 3, or 4 weeks, every 1, 2, 3, 4, 5, 6, 7, 8, 9, 10, or 11 months, or every 1, 2, 3, 4, 5, 6, 7, 8, 9, 10, or more years. For example, after a subject is selected for administration of a treatment for reducing risk of developing a cardiac arrhythmia and the subject is administered a treatment for reducing risk of developing a cardiac arrhythmia, the subject can be monitored at irregular intervals.

Some embodiments further include recording the selection of the subject for administration of a treatment for reducing risk of developing a cardiac arrhythmia in the subject's medical record (e.g., a computer readable medium). Some examples further include informing the subject, the subject's family, and/or the subject's primary care physician or attending physician that the subject has been selected for administration of a treatment for reducing risk of developing a cardiac arrhythmia. Some examples further include informing the subject's insurance provider that the subject has been selected for administration of a treatment for reducing risk of developing a cardiac arrhythmia.

### Methods of Determining the Efficacy of a Treatment in a Subject Having or at Risk of Developing a Cardiac Arrhythmia

Also provided herein are methods of determining the efficacy of a treatment in a subject having or at risk of developing a cardiac arrhythmia (e.g., atrial fibrillation). In some embodiments, the methods include: (a) one or more (e.g., 1, 2, or 3) of: determining the level of CBG in a first sample obtained from a subject at a first time point, determining the level of GC in a first sample obtained from the subject at the first time point, and determining the level of NE in a first sample obtained from the subject at the first time point; (b) administering a treatment to the subject between the first time point and a second time point; (c) one or more (e.g., 1, 2, or 3) of: determining the level of CBG in a second sample obtained from the subject at the second time point, determining the level of GC in a second sample obtained from the subject at the second time point, and determining the level of NE in a second sample obtained from the subject at the second time point; (c) comparing the determined level of one or more (e.g., 1, 2, or 3) of CBG, GC, and NE at the second time point to the determined level of one or more of CBG, GC, and NE at the first time point; and (d) (i) determining that the treatment administered to a subject having one or more (e.g., 1, 2, or 3) of: a determined CBG level at the second time point that is reduced as compared to a determined CBG level at the first time point, a determined GC level at the second time point that is elevated as compared to a determined GC level at the first time point, and a determined NE level at the second time point that is elevated as compared to a determined NE level at the first time point was not effective, or (ii) determining that the treatment administered to a subject having one or more (e.g., 1, 2, or 3) of: a determined CBG level at the second time point that is about the same or increased as compared to a determined CBG level at the first time point, a determined GC level at the second time point that is about the same or decreased as compared to a determined GC level at the first time point, and a determined NE level at the second time point that is about the same or decreased as compared to a determined NE level at the first time point was effective. In some embodiments, step (a), step (c), or both includes performing an assay (e.g., an immunoassay) to determine the level(s) of one or more (e.g., 1, 2, or 3) of CBG, GC, and/or NE in the first sample, the second sample, or both, respectively. Additional exemplary assays for determining the levels of one or more (e.g., 1, 2, or 3) of CBG, GC, and/or NE in the first sample, the second sample, or both are disclosed herein. In some embodiments, step (a), step (c), or both includes determining the level(s) of one or more (e.g., 1, 2, or 3) of CBG, GC, and/or NE in the first sample, the second sample, or both, respectively, where the first sample, the second sample, or both includes blood, plasma, or serum obtained from the subject.

In some embodiments, the methods of determining the efficacy of a treatment in a subject having or at risk of developing a cardiac arrhythmia include determining the level of one of CBG, GC, or NE in a sample at a first time point prior to treatment, and comparing the determined level of CBG, GC, or NE at the first time point to the level of CBG, GC, or NE in a sample at a second time point subsequent to treatment, respectively. In some embodiments, the methods of determining the efficacy of a treatment in a subject having or at risk of developing a cardiac arrhythmia include determining the levels of two of CBG, GC, and NE in a sample (e.g., CBG and GC, CPB and NE, or GC and NE) at a first time point prior to treatment, and comparing the determined level(s) of two of CBG, GC, and NE at the first time point to the levels of two of CBG, GC, and NE in a sample at a second time point subsequent to treatment, respectively. In some embodiments, the methods of determining the efficacy of a treatment in a subject having or at risk of developing a cardiac arrhythmia include determining the levels of each of CBG, GC, and NE in a sample at a first time point prior to treatment, and comparing the determined levels of CBG, GC, and NE at the first time point to the levels of CBG, GC, or NE in a sample at a second time point subsequent to treatment, respectively.

The treatment administered between the first and the second time point can be, e.g., one or more (e.g., 1, 2, 3, 4, 5, 6, 7, 8, 9, 10, or more) of any of the treatments disclosed herein (e.g., any of the treatments for reducing risk of developing a cardiac arrhythmia described herein or known in the art, or any of the treatments for cardiac arrhythmia known in the art). In some embodiments, a subject administered a treatment between the first time point and the second time point in order to reduce the risk of developing a cardiac arrhythmia, delay the onset of a cardiac arrhythmia, or reduce the severity of symptoms of a cardiac arrhythmia in the subject. In some embodiments, a subject can be administered two or more (e.g., 2, 3, 4, 5, 6, 7, 8, 9, 10, or more) treatments (e.g., any of the treatments described herein or known in the art) between the first time point and the second time point, where the two or more treatments are administered between the first time point and the second time point in combination. In some embodiments, a subject can be administered two or more (e.g., 2, 3, 4, 5, 6, 7, 8, 9, 10, or more) treatments (e.g., any of the treatments described herein or known in the art) between the first time point and the second time point, where the two or more treatments are sequentially administered between the first time point and the second time point. In some embodiments, when a subject is administered two or more treatments between the first and second time point, each of the treatments can be of the same general type (e.g., medications, surgical procedures, electrical procedures, etc.). In some embodiments, when a subject is administered two or more treatments between the first and second time point, each of the treatments can be different general types (e.g., beta blockers, procainamide, amiodarone, dronedarone, dofetilide, ibutilide, propafenone, flecainide, anticoagulants, bloodthinners, anti-platelet drugs, cardioversion, ablation, defibrillation, etc.).

In some embodiments, one or more treatments (e.g., any of the treatments described herein) are administered to the subject 1, 2, 3, 4, 5, 6, 7, 8, 9, 10, 15, 20, 25, or 30 days after the first time point (and before the second time point). In some embodiments, one or more treatments are administered to the subject 1, 2, 3, 4, 5, 6, 7, 8, 9, 10, or more weeks after the first time point (and before the second time point).

In some embodiments, the period of time between the first time point and the second time point is 1, 2, 3, 4, 5, 6, 7, 8, 9, 10, 15, 20, 25, or 30 days. In some embodiments, the period of time between the first time point and the second time point is 1, 2, 3, 4, 5, 6, 7, 8, 9, 10, or more weeks.

In some embodiments, the efficacy of a treatment in a subject having or at increased risk of developing a cardiac arrhythmia is determined by determining the level(s) of one or more (e.g., 1, 2, or 3) of CBG, GC, or NE in a sample at multiple time points (e.g., 2, 3, 4, 5, 6, 7, 8, 9, 10 or more time points) after one or more treatments is administered to the subject, and comparing the determined level(s) at a later time point to the determined level(s) at an earlier time point. In some embodiments, the determined level(s) of one or more (e.g., 1, 2, or 3) of CBG, GC, or NE at subsequently determined time points (as compared to the determined level(s) at an earlier time point) indicates that the administered one or more treatments are effective. For example, the determined level(s) of one or more (e.g., 1, 2, or 3) of CBG, GC, or NE over time can indicate that the one or more administered treatments are progressively more effective over time.

In some embodiments, the treatment is administered to the subject for the purpose of reducing the risk of developing a cardiac arrhythmia in the subject, delaying the onset of a cardiac arrhythmia in the subject, or reducing the severity of symptoms of a cardiac arrhythmia in the subject. In some embodiments, a subject can be administered a treatment at a time point (between the first and second time points) when the subject does not present traditional risk factors such as diabetes, obesity, or metabolic syndrome. In some examples, a subject can be administered a treatment at a time point (between the first and second time points) when the subject has a BMI of about 30 or less (e.g., about 29 or less, about 28 or less, about 27 or less, about 26 or less, about 25 or less). In some examples, a subject can be administered a treatment at a time point (between the first and second time points) when the subject does not have an elevated level of troponin C in a sample (e.g., blood, serum, or plasma) obtained from the subject, as compared to a reference level of troponin C. A reference level of troponin C can be, e.g., a level in a subject not presenting with one or more symptoms of cardiac arrhythmia and/or not diagnosed as having cardiac arrhythmia, a level in a healthy subject or a population of healthy subjects, a threshold level, a level in a subject or population of subjects not identified as having a genetic risk (e.g., an elevated genetic risk) of developing cardiac arrhythmia, a level in a sample from a subject or a population of subjects that has/have no history of a cardiac arrhythmia, or a level in a sample from a subject or a population of subjects that does/do not have a genetically-related family member diagnosed or identified as having a cardiac arrhythmia.

In some embodiments, the subject has been previously diagnosed as having a cardiac arrhythmia. In some embodiments, the subject has previously been identified as having an increased risk of developing a cardiac arrhythmia (e.g., using any of the methods described herein).

In some embodiments, after a subject is administered a treatment, the subject can be monitored at periodic intervals, e.g., every 1, 2, 3, 4, 5, or 6 days, every 1, 2, 3, or 4 weeks, every 1, 2, 3, 4, 5, 6, 7, 8, 9, 10, or 11 months, or every 1, 2, 3, 4, 5, 6, 7, 8, 9, 10, or more years. In some embodiments, the subject is monitored at equal periodic intervals after the subject is administered a treatment. In some embodiments, the subject is monitored at irregular intervals after the subject is administered a treatment.

Some embodiments further include recording the determined efficacy of the treatment administered to the subject between the first and second time point in the subject's medical record (e.g., a computer readable medium). Some examples further include informing the subject, the subject's family, and/or the subject's primary care physician or attending physician about the determined efficacy of the treatment administered to the subject between the first and second time point. Some examples further include informing the subject's insurance provider about the determined efficacy of the treatment administered to the subject between the first and second time point.

### Methods of Monitoring a Subject Having or at Increased Risk of Developing a Cardiac Arrhythmia

Also provided herein are methods of monitoring a subject having or at increased risk of developing a cardiac arrhythmia (e.g, atrial fibrillation). In some embodiments, the methods include: (a) one or more (e.g., 1, 2, or 3) of: determining the level of CBG in a first sample obtained from a subject at a first time point, determining the level of GC in a first sample obtained from the subject at the first time point, and determining the level of NE in a first sample obtained from the subject at the first time point; (b) one or more (e.g., 1, 2, or 3) of: determining the level of CBG in a second sample obtained from the subject at a second time point, determining the level of GC in a second sample obtained from a subject at the second time point, and determining the level of NE in a second sample obtained from a subject at the second time point; (c) comparing the determined level of one or more (e.g., 1, 2, or 3) of CBG, GC, and NE at the second time point to the determined level of one or more (e.g., 1, 2, or 3) of CBG, GC, and NE at the first time point; and (d) (i) identifying a subject having one or more (e.g., 1, 2, or 3) of: a determined CBG level at the second time point that is reduced as compared to a determined CBG level at the first time point, a determined GC level at the second time point that is elevated as compared to a determined GC level at the first time point, and a determined NE level at the second time point that is elevated as compared to a determined NE level at the first time point as having a condition that is worsening; (ii) identifying a subject having one or more (e.g., 1, 2, or 3) of: a determined CBG level at the second time point that is increased as compared to a determined CBG level at the first time point, a determined GC level at the second time point that is decreased as compared to a determined GC level at the first time point, and a determined NE level at the second time point that is decreased as compared to a determined NE level at the first time point as having a condition that is improving; or (iii) identifying a subject having one or more (e.g., 1, 2, or 3) of: a determined CBG level at the second time point that is about the same as compared to a determined CBG level at the first time point, a determined GC level at the second time point that is about the same as compared to a determined GC level at the first time point, and a determined NE level at the second time point that is about the same as compared to a determined NE level at the first time point as having a condition that is static. In some embodiments, step (a), step (b), or both includes performing an assay (e.g., an immunoassay) to determine the level(s) of one or more (e.g., 1, 2, or 3) of CBG, GC, and/or NE in a sample obtained from the subject at a first time point, a sample obtained from the subject at a second time point, or both, respectively. Additional exemplary assays for determining the levels of one or more (e.g., 1, 2, or 3) of CBG, GC, and/or NE in a sample obtained from the subject at a first time point, a sample obtained from the subject at a second time point, or both, are disclosed herein. In some embodiments, step (a), step (b), or both includes determining the level(s) of one or more (e.g., 1, 2, or 3) of CBG, GC, and/or NE in a sample obtained from the subject at a first time point, a sample obtained from the subject at a second time point, or both, where the sample obtained from the subject at a first time point, the sample obtained from the subject at a second time point, or both, includes blood, plasma, or serum obtained from the subject.

In some embodiments, the methods of monitoring a subject having or at increased risk of developing a cardiac arrhythmia include determining the level of one of CBG, GC, or NE in a sample obtained from the subject at a first time point, and comparing the determined level of CBG, GC, or NE in the sample obtained at the first time point to the determined level of CBG, GC, or NE in a sample obtained from the subject at a second time point, respectively. In some embodiments, the methods of monitoring a subject having or at increased risk of developing a cardiac arrhythmia include determining the levels of two of CBG, GC, or NE in a sample (e.g., CBG and GC, CPB and NE, or GC and NE) obtained from the subject at a first time point, and comparing the determined levels of two of CBG, GC, or NE in the sample obtained from the subject at the first time point to the determined levels of two of CBG, GC, or NE in a sample obtained from the subject at a second time point, respectively. In some embodiments, the methods of monitoring a subject having or at increased risk of developing a cardiac arrhythmia include determining the levels of each of CBG, GC, and NE in a sample obtained from the subject at a first time point, and comparing lo the determined levels of CBG, GC, and NE in the sample obtained from the subject at the first time point to the determined levels of CBG, GC, or NE in a sample obtained from the subject at a second time point, respectively.

In some embodiments, a monitored subject is administered (e.g., between the first or second time points) or has been previously administered one or more (e.g., 1, 2, 3, 4, 5, 6, 7, 8, 9, 10, or more) treatment(s) (e.g., any of the treatments described herein). In some embodiments, a monitored subject is administered (e.g., between the first and second time points) two or more (e.g., 1, 2, 3, 4, 5, 6, 7, 8, 9, 10, or more) treatments (e.g., any of the treatments described herein) in combination. In some embodiments, a monitored subject is sequentially administered (e.g., between the first and second time points) two or more (e.g., 1, 2, 3, 4, 5, 6, 7, 8, 9, 10, or more) treatments (e.g., any of the treatments described herein).

In some embodiments, a subject having or at increased risk of developing a cardiac arrhythmia who is receiving one or more treatments (e.g., any of the treatments described herein) is monitored according to any of methods described herein. In some embodiments, the subject is determined to have a condition that is worsening. For example, a monitored subject can be determined to have a condition that is worsening when one or more (e.g., 1, 2, or 3) of the following criteria are met: the determined CBG level in the sample obtained from the subject at the second time point is reduced as compared to the determined CBG level in the sample obtained from the subject at the first time point, the determined GC level in the sample obtained from the subject at the second time point is elevated as compared to the determined GC level in the sample obtained from the subject at the first time point, and the determined NE level in the sample obtained from the subject at the second time point is elevated as compared to the determined NE level in a sample obtained from the subject at the first time point. In some embodiments, a subject who is receiving or has received one or more treatments and is determined to have a condition that is worsening (e.g., using any of the methods described herein) can be administered one or more (e.g., 1, 2, 3, 4, 5, 6, 7, 8, 9, 10, or more) alternative treatments that are different from the treatment(s) the subject is receiving or has received. In some embodiments, after the subject has received one or more alternative treatments, the efficacy of such treatments can be determined according to methods disclosed herein.

In some embodiments, the subject is determined to have a condition that is improving. For example, a monitored subject can be determined to have a condition that is improving when one or more (e.g., 1, 2, or 3) of the following criteria are met: the determined CBG level in the sample obtained from the subject at the second time point is elevated as compared to the determined CBG level in the sample obtained from the subject at the first time point, the determined GC level in the sample obtained from the subject at the second time point is reduced as compared to the determined GC level in the sample obtained from the subject at the first time point, and the determined NE level in the sample obtained from the subject at the second time point is reduced as compared to the determined NE level in the sample obtained from the subject at the first time point. In some embodiments, a subject who is receiving or has received one or more treatments and is determined to have a condition that is improving can be administered additional doses of the treatment(s). In some embodiments, a subject who is receiving or has received one or more treatments and is determined to have a condition that is improving can be administered one or more (e.g., 1, 2, 3, 4, 5, 6, 7, 8, 9, 10, or more) alternative treatments in combination with the treatments that the subject is receiving or has previously received. In some embodiments, after the subject has received one or more alternative treatments, the efficacy of such treatments can be determined according to methods disclosed herein.

In some embodiments, the subject is determined to have a condition that is static. For example, a monitored subject can be determined to have a condition that is static when one or more (e.g., 1, 2, or 3) of the following criteria are met: the determined CBG level in the sample obtained from the subject at the second time point is about the same as compared to the determined CBG level in the sample obtained from the subject at the first time point, the determined GC level in the sample obtained from the subject at the second time point is about the same as compared to the determined GC level in the sample obtained from the subject at the first time point, and the determined NE level in the sample obtained from the subject at the second time point is about the same as compared to the determined NE level in the sample obtained from the subject at the first time point. In some embodiments, a subject who is receiving or has received one or more treatments and is determined to have a condition that is static can be administered additional doses of the treatment(s). In some embodiments, a subject who is receiving or has received one or more treatments and is determined to have a condition that is static can be administered one or more (e.g., 1, 2, 3, 4, 5, 6, 7, 8, 9, 10, or more) alternative treatments in combination with the treatments the subject is currently receiving or has received. In some embodiments, after the subject has received one or more alternative treatments, the efficacy of such treatments can be determined according to methods disclosed herein.

In some embodiments, the period of time between the first time point and the second time point is 1, 2, 3, 4, 5, 6, 7, 8, 9, 10, 15, 20, 25, or 30 days. In some embodiments, the period of time between the first time point and the second time point is 1, 2, 3, 4, 5, 6, 7, 8, 9, 10, or more weeks.

In some embodiments, the methods of monitoring are performed prior to observing or detecting conventional symptoms or predictive indicators of cardiac arrhythmia in the subject. In some examples, the subject does not present with traditional risk factors such as diabetes, obesity, or metabolic syndrome. In some examples, the subject has a BMI of about 30 or less (e.g., about 29 or less, about 28 or less, about 27 or less, about 26 or less, about 25 or less). In some examples, the subject does not have an elevated level of troponin C in a sample (e.g., blood, serum, or plasma) obtained from the subject, as compared to a reference level of troponin C. A reference level of troponin C can be, e.g., a level in a subject not presenting with one or more symptoms of cardiac arrhythmia and/or not diagnosed as having cardiac arrhythmia, a level in a healthy subject or a population of healthy subjects, a threshold level, a level in a subject or population of subjects not identified as having a genetic risk (e.g., an elevated genetic risk) of developing cardiac arrhythmia, a level in a sample from a subject or a population of subjects that has/have no history of a cardiac arrhythmia, or a level in a sample from a subject or a population of subjects that does/do not have a genetically-related family member diagnosed or identified as having a cardiac arrhythmia.

In some embodiments, a subject has been previously diagnosed as having a cardiac arrhythmia. In some embodiments, a subject has been previously identified as having an increased risk of developing a cardiac arrhythmia (e.g., using any of the methods described herein).

Some embodiments further include recording the subject's determined condition in the subject's medical record (e.g., a computer readable medium). Some examples further include informing the subject, the subject's family, and/or the subject's primary care physician or attending physician about the subject's determined condition. Some examples further include informing the subject's insurance provider about the subject's determined condition.

### Kits

Also provided herein are kits that can be used to perform any of the methods described herein. In some embodiments, these kits include one or more (e.g., 1, 2, or 3) reagents for detecting a level(s) of one or more (e.g., 1, 2, or 3) of CBG, GC, and/or NE in a sample. In some embodiments, kits provided herein include samples containing reference levels of one or more (e.g., 1, 2, or 3) of CBG, GC, and/or NE.

In some examples, the kits provided herein include two or more (e.g., 2 or 3) antibodies for detecting a level(s) of one or more (e.g., 1, 2, or 3) of CBG, GC, and/or NE in a sample. For example, a kit can include two or more of: (a) an antibody that binds specifically to CBG (b) an antibody that binds specifically to GC, and (c) an antibody that binds specifically to NE. In some embodiments, a kit can include components for detecting a level(s) of one or more (e.g., 1, 2, or 3) of CBG, GC, and/or NE in a sample in an ELISA, an antigen capture assay, a quantitative Western blotting assay, a radioimmunoassays (RIA), a flow cytometry assay, a quantum dot assay, a protein-based microarray, and/or a quantitative mass spectrometry method, e.g., a SELDI-TOF mass spectrometry method. In some embodiments, a kit can include components for detecting one or more (e.g., 1, 2, or 3) of CBG, GC, and/or NE nucleic acids in a sample in a Northern blot assay, a quantitative RT-PCR assay, a nuclease protection assay, and/or a microarray assay. In some embodiments, kits further include instructions for performing any of the methods described herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining a subject's risk of developing a cardiac arrhythmia, the method comprising:
(a) one or more of:
performing an immunoassay to determine the level of corticosteroid-binding globulin (CBG) in a sample comprising blood, plasma, or serum obtained from a subject;
performing an immunoassay to determine the level of glucocorticoid (GC) in a sample comprising blood, plasma, or serum obtained from the subject; and
performing an immunoassay to determine the level of neutrophil elastase (NE) in a sample comprising blood, plasma, or serum obtained from the subject;
(b) comparing the determined level(s) of one or more of CBG, GC, and NE to reference level(s) of CBG, GC, and NE, respectively; and
(c) identifying a subject having one or more of a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia; or
identifying a subject having one or more of a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia.

2. The method of claim 1, wherein step (c) comprises identifying a subject having one or more of a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia.

3. The method of claim 2, wherein step (c) comprises identifying a subject having two or three of a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia.

4. The method of claim 3, wherein step (c) comprises identifying a subject having a determined level of CBG that is decreased as compared to a reference level of CBG, a determined level of GC that is elevated as compared to a reference level of GC, and a determined level of NE that is elevated as compared to a reference level of NE as having an increased risk of developing a cardiac arrhythmia.

5. The method of claim 1, wherein step (c) comprises identifying a subject having one or more of a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia.

6. The method of claim 5, wherein step (c) comprises identifying a subject having two or three of a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia.

7. The method of claim 6, wherein step (c) comprises identifying a subject having a determined level of CBG that is about the same or elevated as compared to a reference level of CBG, a determined level of GC that is about the same or decreased as compared to a reference level of GC, and a determined level of NE that is about the same or decreased as compared to a reference level of NE as having a decreased risk of developing a cardiac arrhythmia.

8-14. (canceled)

15. A method of treating a subject, the method comprising:
(a) one or more of:
performing an immunoassay to determine the level of corticosteroid-binding globulin (CBG) in a sample comprising blood, plasma, or serum obtained from a subject;
performing an immunoassay to determine the level of glucocorticoid (GC) in a sample comprising blood, plasma, or serum obtained from the subject; and
performing an immunoassay to determine the level of neutrophil elastase (NE) in a sample comprising blood, plasma, or serum obtained from the subject;
(b) comparing the determined level(s) of one or more of CBG, GC, and NE to reference level(s) of CBG, GC, and NE, respectively;
(c) selecting a subject having one or more of a determined level of CBG that is decreased as compared to a reference level of CBG, a determined level of GC that is elevated as compared to a reference level of GC, and a determined level of NE that is elevated as compared to a reference level of NE; and
(d) administering a treatment for reducing risk of developing a cardiac arrhythmia to the selected subject.

16. The method of claim 15, wherein step (c) comprises selecting a subject having two or three of a determined level of CBG that is decreased as compared to a reference level of CBG, a determined level of GC that is elevated as compared to a reference level of GC, and a determined level of NE that is elevated as compared to a reference level of NE.

17. The method of claim 16, wherein step (c) comprises selecting a subject having a determined level of CBG that is decreased as compared to a reference level of CBG, a determined level of GC that is elevated as compared to a reference level of GC, and a determined level of NE that is elevated as compared to a reference level of NE.

18-20. (canceled)

21. A method of selecting a treatment for reducing risk of developing a cardiac arrhythmia for a subject, the method comprising:
(a) one or more of:
performing an immunoassay to determine the level of corticosteroid-binding globulin (CBG) in a sample comprising blood, plasma, or serum obtained from a subject;
performing an immunoassay to determine the level of glucocorticoid (GC) in a sample comprising blood, plasma, or serum obtained from the subject; and
performing an immunoassay to determine the level of neutrophil elastase (NE) in a sample comprising blood, plasma, or serum obtained from the subject;
(b) comparing the determined level(s) of one or more of CBG, GC, and NE to reference level(s) of CBG, GC, and NE, respectively; and
(c) selecting a treatment for reducing risk of developing a cardiac arrhythmia for a subject having one or more of a determined level of CBG that is decreased as compared to a reference level of CBG, a determined level of GC that is elevated as compared to a reference level of GC, and a determined level of NE that is elevated as compared to a reference level of NE.

22-28. (canceled)

29. A method of selecting a subject for administration of a treatment for reducing risk of developing a cardiac arrhythmia, the method comprising:
(a) one or more of:
performing an immunoassay to determine the level of corticosteroid-binding globulin (CBG) in a sample comprising blood, plasma, or serum obtained from a subject;
performing an immunoassay to determine the level of glucocorticoid (GC) in a sample comprising blood, plasma, or serum obtained from the subject; and
performing an immunoassay to determine the level of neutrophil elastase (NE) in a sample comprising blood, plasma, or serum obtained from the subject;
(b) comparing the determined level(s) of one or more of CBG, GC, and NE to reference level(s) of CBG, GC, and NE, respectively; and
(c) selecting a subject having one or more of a determined level of CBG that is decreased as compared to a reference level of CBG, a determined level of GC that is elevated as compared to a reference level of GC, and a determined level of NE that is elevated as compared to a reference level of NE for administration of a treatment for reducing the risk of developing a cardiac arrhythmia.

30-36. (canceled)

37. The method of claim 15, wherein the treatment for reducing risk of developing a cardiac arrhythmia is selected from the group consisting of: a selective serotonin reuptake inhibitor (SSRI), a norephinephrine reuptake inhibitor (NRI), a dopamine reuptake inhibitor (DRI), a serotonin/norephinephrine reuptake inhibitor (SNRI), a norepinephrine/dopamine reuptake inhibitor (NDRI), a triple reuptake inhibitor (TM), an anti-clotting medication, a heart rate management medication, a heart rhythm management medication, a blood pressure management medication, a neutrophil elastase inhibitor, a cholesterol management medication, a cortisol management medication, a sympathetic activity management medication, non-drug treatments, a surgical procedure, and increased monitoring.

38. The method of claim 1, wherein the subject does not have diabetes or metabolic syndrome.

39. The method of claim 1, wherein the subject has a BMI of ≤30.

40. (canceled)

41. The method of claim 1, wherein the subject does not have an elevated level of troponin C in a sample comprising blood, serum, or plasma sample obtained from the subject, as compared to a reference level of troponin C.

42. The method of claim 41, wherein the reference level of troponin C is the level of troponin C present in a healthy subject or a population of healthy subjects, or a subject or a population of subjects determined to have a low risk of developing a cardiac arrhythmia.

43-48. (canceled)

49. A method of determining the efficacy of a treatment in a subject having or at increased risk of developing a cardiac arrhythmia, the method comprising:
(a) one or more of:
performing an immunoassay to determine the level of corticosteroid-binding globulin (CBG) in a first sample comprising blood, plasma, or serum obtained from a subject at a first time point;
performing an immunoassay to determine the level of glucocorticoid (GC) in a first sample comprising blood, plasma, or serum obtained from the subject at the first time point; and
performing an immunoassay to determine the level of neutrophil elastase (NE) in a first sample comprising blood, plasma, or serum obtained from the subject at the first time point;
(b) administering a treatment to the subject between the first time point and a second time point;
(c) one or more of:
performing an immunoassay to determine the level of CBG in a second sample comprising blood, plasma, or serum obtained from the subject at the second time point;
performing an immunoassay to determine the level of GC in a second sample comprising blood, plasma, or serum obtained from the subject at the second time point; and
performing an immunoassay to determine the level of NE in a second sample comprising blood, plasma, or serum obtained from the subject at the second time point;
(c) comparing the determined level of one or more of CBG, GC, and NE at the second time point to the determined level of one or more of CBG, GC, and NE at the first time point; and
(d) determining that the treatment administered to a subject having one or more of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point was not effective; or
determining that the treatment administered to a subject having one or more of: a determined CBG level at the second time point that is about the same or increased as compared to a determined CBG level at the first time point; a determined GC level at the second time point that is about the same or decreased as compared to a determined GC level at the first time point; and a determined NE level at the second time point that is about the same or decreased as compared to a determined NE level at the first time point was effective.

50-64. (canceled)

65. A method of monitoring a subject having or at increased risk of developing a cardiac arrhythmia, the method comprising:
(a) one or more of:
performing an immunoassay to determine the level of corticosteroid-binding globulin (CBG) in a first sample comprising blood, plasma, or serum obtained from a subject at a first time point;
performing an immunoassay to determine the level of glucocorticoid (GC) in a first sample comprising blood, plasma, or serum obtained from the subject at the first time point; and
performing an immunoassay to determine the level of neutrophil elastase (NE) in a first sample comprising blood, plasma, or serum obtained from the subject at the first time point;
(b) one or more of:
performing an immunoassay to determine the level of CBG in a second sample comprising blood, plasma, or serum obtained from the subject at a second time point;
performing an immunoassay to determine the level of GC in a second sample comprising blood, plasma, or serum obtained from a subject at the second time point; and
performing an immunoassay to determine the level of NE in a second sample comprising blood, plasma, or serum obtained from a subject at the second time point;
(c) comparing the determined level of one or more of CBG, GC, and NE at the second time point to the determined level of one or more of CBG, GC, and NE at the first time point; and
(d) identifying a subject having one or more of: a determined CBG level at the second time point that is reduced as compared to a determined CBG level at the first time point; a determined GC level at the second time point that is elevated as compared to a determined GC level at the first time point; and a determined NE level at the second time point that is elevated as compared to a determined NE level at the first time point as having a condition that is worsening;
identifying a subject having one or more of: a determined CBG level at the second time point that is increased as compared to a determined CBG level at the first time point; a determined GC level at the second time point that is decreased as compared to a determined GC level at the first time point; and a determined NE level at the second time point that is decreased as compared to a determined NE level at the first time point as having a condition that is improving; or
identifying a subject having one or more of: a determined CBG level at the second time point that is about the same as compared to a determined CBG level at the first time point; a determined GC level at the second time point that is about the same as compared to a determined GC level at the first time point; and a determined NE level at the second time point that is about the same as compared to a determined NE level at the first time point as having a condition that is static.

66-92. (canceled)

93. The method of claim 1, wherein the reference level of CBG is the level of CBG in a healthy subject or a population of healthy subjects, or a subject or a population of subjects determined to have a low risk of developing a cardiac arrhythmia.

94. The method of claim 1, wherein the reference level of GC is the level of GC in a healthy subject or a population of healthy subjects, or in a subject or a population of subjects determined to have a low risk of developing a cardiac arrhythmia.

95. The method of claim 1, wherein the reference level of NE is the level of NE in a healthy subject or a population of healthy subjects, or in a subject or a population of subjects determined to have a low risk of developing a cardiac arrhythmia.

96. (canceled)
